SEQUENCE LISTING (44 sequences)

SEQ ID NO: 1 - DNA, 638 bp, Mus musculus
  source 1..638: mol_type = other DNA
gcatgccaatttatagtgccgtcactaacagtactgatactttaacatgctaagtttaaagtgtgtgctatattaattgtaagattggtgaagagaggtgttatcagatggaagctgcacatttctggattaatgtggttaaatgtatcttctcctgtgattactgtctttatttcttcttttaaaatattgtcatttggacatctatctgtatagctacgccctgacacgtcctcctggagacagataagaagttacgacgggaggagcagatggaggcaaagcgctgtgatgcttttgtggtttgagtgcacacatttgttcagtgattctgtgaaatgagtgagcaaatggtgaccgggtgccctgtaaatggtgttctacatcttaagagaagaacacggacactaggtaagtgaagcttgctgtcactcctctacagcgtcacagagggtcagtcacccttgaccacactgaactagtcgtcacctttccactcttcctgccagaagagcagaaatcagactctctggggatatcagcctcacccctactgctctctccattatgaggcaaactttctttcacttcccagaggctctgggggcagcaaggtcaaccctttcctcagactctag

SEQ ID NO: 2 - DNA, 335 bp, Mus musculus
  source 1..335: mol_type = other DNA
tctcggaggagatcagatcgcgcttattcaagggaaccagcccctgctctgcgccctggtccaaggctgttgaagagtgacaaaaggcaccacgctgcggggacgcgggtgaagcccctctgtgtgtcctctgggcataatcaggaactggtgccaaatcagaggtgatgtggccagggctttgggagtgacgcgcggctgggaggcttgcgcacccaaggcacgcccctgccaagtcccactagcagctctttggagacctgggccggctcagccacttcccccagtccctcctccggcaaggggctatatagatctcccaggtcagggcgcag

SEQ ID NO: 3 - DNA, 973 bp, synthetic construct
  misc_feature 1..973: note = Laboratory-synthesized seqeunce.
  source 1..973: mol_type = other DNA
gcatgccaatttatagtgccgtcactaacagtactgatactttaacatgctaagtttaaagtgtgtgctatattaattgtaagattggtgaagagaggtgttatcagatggaagctgcacatttctggattaatgtggttaaatgtatcttctcctgtgattactgtctttatttcttcttttaaaatattgtcatttggacatctatctgtatagctacgccctgacacgtcctcctggagacagataagaagttacgacgggaggagcagatggaggcaaagcgctgtgatgcttttgtggtttgagtgcacacatttgttcagtgattctgtgaaatgagtgagcaaatggtgaccgggtgccctgtaaatggtgttctacatcttaagagaagaacacggacactaggtaagtgaagcttgctgtcactcctctacagcgtcacagagggtcagtcacccttgaccacactgaactagtcgtcacctttccactcttcctgccagaagagcagaaatcagactctctggggatatcagcctcacccctactgctctctccattatgaggcaaactttctttcacttcccagaggctctgggggcagcaaggtcaaccctttcctcagactctagtctcggaggagatcagatcgcgcttattcaagggaaccagcccctgctctgcgccctggtccaaggctgttgaagagtgacaaaaggcaccacgctgcggggacgcgggtgaagcccctctgtgtgtcctctgggcataatcaggaactggtgccaaatcagaggtgatgtggccagggctttgggagtgacgcgcggctgggaggcttgcgcacccaaggcacgcccctgccaagtcccactagcagctctttggagacctgggccggctcagccacttcccccagtccctcctccggcaaggggctatatagatctcccaggtcagggcgcag

SEQ ID NO: 4 - DNA, 295 bp, Rattus norvegicus
  source 1..295: mol_type = other DNA
gacgtcacagtgggtcagtcacccttgatcacactgcaccagtcttcacctttccacgcttcctgccagagcatgaatcaggctctctggggataccggcctcacccctactgaggcaaactttctcccacttctcagaggctctgagggcagcaaggtcagccctttctttggaatctagaaccactccctgtcttgagctgacatcacagggcaggcagatgcagcagggaagggcctgggactgggacgttcatcctacaagaaagctgtggaacttttcagcaacatctca

SEQ ID NO: 5 - DNA, 427 bp, Rattus norvegicus
  source 1..427: mol_type = other DNA
gaaatcagatcgcacttattcaaaggagccaggccctgctctgcgccctggtggaggctcctcatgtgaagagtgacaaaaggcaccatgttgtggatacggggcgaagcccctccggtgtgtcctccaggcatcatcaggaactagtgccaaagcagaggtgctggccagggctttgggagtgacgcgcgtctgggaggcttgtgcgcccagggcacgcccctgccgattcccactagcaggtcttgggggacctgggccggctctgcccctcctccagcaatcgggctataaagctcttccaagtcagggcgcagaagtgccgggcgatccgggcttaaagagcgagaggaagggacgctcacctttgagctcctccacaaatagccctggtggctgccacagaagttcgaagttgagagttcgg

SEQ ID NO: 6 - DNA, 722 bp, synthetic construct
  misc_feature 1..722: note = Laboratory-synthesized seqeunce
  source 1..722: mol_type = other DNA
gacgtcacagtgggtcagtcacccttgatcacactgcaccagtcttcacctttccacgcttcctgccagagcatgaatcaggctctctggggataccggcctcacccctactgaggcaaactttctcccacttctcagaggctctgagggcagcaaggtcagccctttctttggaatctagaaccactccctgtcttgagctgacatcacagggcaggcagatgcagcagggaagggcctgggactgggacgttcatcctacaagaaagctgtggaacttttcagcaacatctcagaaatcagatcgcacttattcaaaggagccaggccctgctctgcgccctggtggaggctcctcatgtgaagagtgacaaaaggcaccatgttgtggatacggggcgaagcccctccggtgtgtcctccaggcatcatcaggaactagtgccaaagcagaggtgctggccagggctttgggagtgacgcgcgtctgggaggcttgtgcgcccagggcacgcccctgccgattcccactagcaggtcttgggggacctgggccggctctgcccctcctccagcaatcgggctataaagctcttccaagtcagggcgcagaagtgccgggcgatccgggcttaaagagcgagaggaagggacgctcacctttgagctcctccacaaatagccctggtggctgccacagaagttcgaagttgagagttcgg

SEQ ID NO: 7 - DNA, 334 bp, Homo sapiens
  source 1..334: mol_type = other DNA
tgatcaagtgcatttgttaatgtgttctacattttcaaaaaggaaaggagaatttgttacattcagaacttgctgccactcctttgctacgtcataaagggtcagttgcccttgctcatactgacctattctttacctctctgcttcttctttgtgccagaagagtagaaatctgaccctttggggataccaccctctcccctactgctctctccaacctgaggcaaactttctcctacttcccagagcctgtcagaagtggtgaagccagcctgctccttggaatccagaactactttcagaatcttgaacttctgtgacctctcagggtccc

SEQ ID NO: 8 - DNA, 289 bp, Homo sapiens
  source 1..289: mol_type = other DNA
accgccgcggtgcgccctccctccgacgtgcggtgtgcggggcgcagacaaccagcggccggcccagggctttcggggagcgaagcagggctcccgaggcaccgagcgagaatgggaatgggagggacccggtgctcccggacacgcccccggcaggtcccacgcccgggtcttctgagacctcgcgcggcccagcccgggagcggcccagctatataagtcccagcggaagaccggaacgcagagggtcctgctggcgcgagggtgggtaggaggggacgcggggact

SEQ ID NO: 9 - DNA, 623 bp, Homo sapiens
  source 1..623: mol_type = other DNA
tgatcaagtgcatttgttaatgtgttctacattttcaaaaaggaaaggagaatttgttacattcagaacttgctgccactcctttgctacgtcataaagggtcagttgcccttgctcatactgacctattctttacctctctgcttcttctttgtgccagaagagtagaaatctgaccctttggggataccaccctctcccctactgctctctccaacctgaggcaaactttctcctacttcccagagcctgtcagaagtggtgaagccagcctgctccttggaatccagaactactttcagaatcttgaacttctgtgacctctcagggtcccaccgccgcggtgcgccctccctccgacgtgcggtgtgcggggcgcagacaaccagcggccggcccagggctttcggggagcgaagcagggctcccgaggcaccgagcgagaatgggaatgggagggacccggtgctcccggacacgcccccggcaggtcccacgcccgggtcttctgagacctcgcgcggcccagcccgggagcggcccagctatataagtcccagcggaagaccggaacgcagagggtcctgctggcgcgagggtgggtaggaggggacgcggggact

SEQ ID NO: 10 - DNA, 888 bp, Homo sapiens
  source 1..888: mol_type = other DNA
atgctatttatgaagatggacctgttgaactatcagtacttggacaagatgaacaacaatatcggcattctgtgctacgaaggcgaagctgctctcaggggagaacccagaatacagaccctgccggtggcctctgccctcagcagtcaccgcaccggccctcccccaatcagccccagcaagaggaagttcagcatggagccaggtgacgaggacctagactgtgacaacgaccacgtctccaaaatgagtcgcatcttcaacccccatctgaacaagactgccaatggagactgccgcagagacccccgggagcggagccgcagccccatcgagcgcgctgtggcccccaccatgagcctgcacggcagccacctgtacacctccctccccagccttggcctggagcagcccctcgcactgaccaagaacagcctggacgccagcaggccagccggcctctcgcccacactgaccccgggggagcggcagcagaaccggccctccgtgatcacctgtgcctcggctggcgcccgcaactgcaacctctcgcactgccccatcgcgcacagcggctgtgccgcgcccgggcctgccagctaccggaggccaccgagcgctgccaccacctgtgaccccgtggtggaggagcatttccgcaggagcctgggcaagaattacaaggagcccgagccggcacccaactccgtgtccatcacgggctccgtggacgaccactttgccaaagctctgggtgacacgtggctccagatcaaagcggccaaggacggagcatccagcagccctgagtccgcctctcgcaggggccagcccgccagcccctctgcccacatggtcagccacagtcactccccctctgtggtctcc

SEQ ID NO: 11 - protein, 296 aa, Homo sapiens
MLFMKMDLLNYQYLDKMNNNIGILCYEGEAALRGEPRIQTLPVASALSSHRTGPPPISPSKRKFSMEPGDEDLDCDNDHVSKMSRIFNPHLNKTANGDCRRDPRERSRSPIERAVAPTMSLHGSHLYTSLPSLGLEQPLALTKNSLDASRPAGLSPTLTPGERQQNRPSVITCASAGARNCNLSHCPIAHSGCAAPGPASYRRPPSAATTCDPVVEEHFRRSLGKNYKEPEPAPNSVSITGSVDDHFAKALGDTWLQIKAAKDGASSSPESASRRGQPASPSAHMVSHSHSPSVVS

SEQ ID NO: 12 - DNA, 870 bp, Homo sapiens
  source 1..870: mol_type = other DNA
atggagacgccattggatgttttgtccagggcagcatctctggtgcatgctgatgacgaaaaacgcgaagctgctctcaggggagaacccagaatacagaccctgccggtggcctctgccctcagcagtcaccgcaccggccctcccccaatcagccccagcaagaggaagttcagcatggagccaggtgacgaggacctagactgtgacaacgaccacgtctccaaaatgagtcgcatcttcaacccccatctgaacaagactgccaatggagactgccgcagagacccccgggagcggagccgcagccccatcgagcgcgctgtggcccccaccatgagcctgcacggcagccacctgtacacctccctccccagccttggcctggagcagcccctcgcactgaccaagaacagcctggacgccagcaggccagccggcctctcgcccacactgaccccgggggagcggcagcagaaccggccctccgtgatcacctgtgcctcggctggcgcccgcaactgcaacctctcgcactgccccatcgcgcacagcggctgtgccgcgcccgggcctgccagctaccggaggccaccgagcgctgccaccacctgtgaccccgtggtggaggagcatttccgcaggagcctgggcaagaattacaaggagcccgagccggcacccaactccgtgtccatcacgggctccgtggacgaccactttgccaaagctctgggtgacacgtggctccagatcaaagcggccaaggacggagcatccagcagccctgagtccgcctctcgcaggggccagcccgccagcccctctgcccacatggtcagccacagtcactccccctctgtggtctcc

SEQ ID NO: 13 - protein, 290 aa, Homo sapiens
METPLDVLSRAASLVHADDEKREAALRGEPRIQTLPVASALSSHRTGPPPISPSKRKFSMEPGDEDLDCDNDHVSKMSRIFNPHLNKTANGDCRRDPRERSRSPIERAVAPTMSLHGSHLYTSLPSLGLEQPLALTKNSLDASRPAGLSPTLTPGERQQNRPSVITCASAGARNCNLSHCPIAHSGCAAPGPASYRRPPSAATTCDPVVEEHFRRSLGKNYKEPEPAPNSVSITGSVDDHFAKALGDTWLQIKAAKDGASSSPESASRRGQPASPSAHMVSHSHSPSVVS

SEQ ID NO: 14 - DNA, 630 bp, Homo sapiens
  source 1..630: mol_type = other DNA
atgattaaagtgaggaacaagactgccaatggagactgccgcagagacccccgggagcggagccgcagccccatcgagcgcgctgtggcccccaccatgagcctgcacggcagccacctgtacacctccctccccagccttggcctggagcagcccctcgcactgaccaagaacagcctggacgccagcaggccagccggcctctcgcccacactgaccccgggggagcggcagcagaaccggccctccgtgatcacctgtgcctcggctggcgcccgcaactgcaacctctcgcactgccccatcgcgcacagcggctgtgccgcgcccgggcctgccagctaccggaggccaccgagcgctgccaccacctgtgaccccgtggtggaggagcatttccgcaggagcctgggcaagaattacaaggagcccgagccggcacccaactccgtgtccatcacgggctccgtggacgaccactttgccaaagctctgggtgacacgtggctccagatcaaagcggccaaggacggagcatccagcagccctgagtccgcctctcgcaggggccagcccgccagcccctctgcccacatggtcagccacagtcactccccctctgtggtctcc

SEQ ID NO: 15 - protein, 210 aa, Homo sapiens
MIKVRNKTANGDCRRDPRERSRSPIERAVAPTMSLHGSHLYTSLPSLGLEQPLALTKNSLDASRPAGLSPTLTPGERQQNRPSVITCASAGARNCNLSHCPIAHSGCAAPGPASYRRPPSAATTCDPVVEEHFRRSLGKNYKEPEPAPNSVSITGSVDDHFAKALGDTWLQIKAAKDGASSSPESASRRGQPASPSAHMVSHSHSPSVVS

SEQ ID NO: 16 - DNA, 618 bp, Homo sapiens
  source 1..618: mol_type = other DNA
atgaacaagactgccaatggagactgccgcagagacccccgggagcggagccgcagccccatcgagcgcgctgtggcccccaccatgagcctgcacggcagccacctgtacacctccctccccagccttggcctggagcagcccctcgcactgaccaagaacagcctggacgccagcaggccagccggcctctcgcccacactgaccccgggggagcggcagcagaaccggccctccgtgatcacctgtgcctcggctggcgcccgcaactgcaacctctcgcactgccccatcgcgcacagcggctgtgccgcgcccgggcctgccagctaccggaggccaccgagcgctgccaccacctgtgaccccgtggtggaggagcatttccgcaggagcctgggcaagaattacaaggagcccgagccggcacccaactccgtgtccatcacgggctccgtggacgaccactttgccaaagctctgggtgacacgtggctccagatcaaagcggccaaggacggagcatccagcagccctgagtccgcctctcgcaggggccagcccgccagcccctctgcccacatggtcagccacagtcactccccctctgtggtctcc

SEQ ID NO: 17 - protein, 206 aa, Homo sapiens
MNKTANGDCRRDPRERSRSPIERAVAPTMSLHGSHLYTSLPSLGLEQPLALTKNSLDASRPAGLSPTLTPGERQQNRPSVITCASAGARNCNLSHCPIAHSGCAAPGPASYRRPPSAATTCDPVVEEHFRRSLGKNYKEPEPAPNSVSITGSVDDHFAKALGDTWLQIKAAKDGASSSPESASRRGQPASPSAHMVSHSHSPSVVS

SEQ ID NO: 18 - DNA, 885 bp, Homo sapiens
  source 1..885: mol_type = other DNA
atgactgagaatacgcattttgacaaaatccctgagtcctgtgcactcaaaagttggagacatccaggtctgcaccatggcgaagctgctctcaggggagaacccagaatacagaccctgccggtggcctctgccctcagcagtcaccgcaccggccctcccccaatcagccccagcaagaggaagttcagcatggagccaggtgacgaggacctagactgtgacaacgaccacgtctccaaaatgagtcgcatcttcaacccccatctgaacaagactgccaatggagactgccgcagagacccccgggagcggagccgcagccccatcgagcgcgctgtggcccccaccatgagcctgcacggcagccacctgtacacctccctccccagccttggcctggagcagcccctcgcactgaccaagaacagcctggacgccagcaggccagccggcctctcgcccacactgaccccgggggagcggcagcagaaccggccctccgtgatcacctgtgcctcggctggcgcccgcaactgcaacctctcgcactgccccatcgcgcacagcggctgtgccgcgcccgggcctgccagctaccggaggccaccgagcgctgccaccacctgtgaccccgtggtggaggagcatttccgcaggagcctgggcaagaattacaaggagcccgagccggcacccaactccgtgtccatcacgggctccgtggacgaccactttgccaaagctctgggtgacacgtggctccagatcaaagcggccaaggacggagcatccagcagccctgagtccgcctctcgcaggggccagcccgccagcccctctgcccacatggtcagccacagtcactccccctctgtggtctcc

SEQ ID NO: 19 - protein, 295 aa, Homo sapiens
MTENTHFDKIPESCALKSWRHPGLHHGEAALRGEPRIQTLPVASALSSHRTGPPPISPSKRKFSMEPGDEDLDCDNDHVSKMSRIFNPHLNKTANGDCRRDPRERSRSPIERAVAPTMSLHGSHLYTSLPSLGLEQPLALTKNSLDASRPAGLSPTLTPGERQQNRPSVITCASAGARNCNLSHCPIAHSGCAAPGPASYRRPPSAATTCDPVVEEHFRRSLGKNYKEPEPAPNSVSITGSVDDHFAKALGDTWLQIKAAKDGASSSPESASRRGQPASPSAHMVSHSHSPSVVS

SEQ ID NO: 20 - DNA, 693 bp, Homo sapiens
  source 1..693: mol_type = other DNA
atggagccaggtgacgaggacctagactgtgacaacgaccacgtctccaaaatgagtcgcatcttcaacccccatctgaacaagactgccaatggagactgccgcagagacccccgggagcggagccgcagccccatcgagcgcgctgtggcccccaccatgagcctgcacggcagccacctgtacacctccctccccagccttggcctggagcagcccctcgcactgaccaagaacagcctggacgccagcaggccagccggcctctcgcccacactgaccccgggggagcggcagcagaaccggccctccgtgatcacctgtgcctcggctggcgcccgcaactgcaacctctcgcactgccccatcgcgcacagcggctgtgccgcgcccgggcctgccagctaccggaggccaccgagcgctgccaccacctgtgaccccgtggtggaggagcatttccgcaggagcctgggcaagaattacaaggagcccgagccggcacccaactccgtgtccatcacgggctccgtggacgaccactttgccaaagctctgggtgacacgtggctccagatcaaagcggccaaggacggagcatccagcagccctgagtccgcctctcgcaggggccagcccgccagcccctctgcccacatggtcagccacagtcactccccctctgtggtctcc

SEQ ID NO: 21 - protein, 231 aa, Homo sapiens
MEPGDEDLDCDNDHVSKMSRIFNPHLNKTANGDCRRDPRERSRSPIERAVAPTMSLHGSHLYTSLPSLGLEQPLALTKNSLDASRPAGLSPTLTPGERQQNRPSVITCASAGARNCNLSHCPIAHSGCAAPGPASYRRPPSAATTCDPVVEEHFRRSLGKNYKEPEPAPNSVSITGSVDDHFAKALGDTWLQIKAAKDGASSSPESASRRGQPASPSAHMVSHSHSPSVVS

SEQ ID NO: 22 - DNA, 888 bp, synthetic construct
  misc_feature 1..888: note = Laboratory-synthesized seqeunce
  source 1..888: mol_type = other DNA
atgctatttatgaagatggacctgttgaactatcagtacttggacaagatgaacaacaatatcggcattctgtgctacgaaggcgaagctgctctcaggggagaacccagaatacagaccctgccggtggcctctgccctcagcagtcaccgcaccggccctcccccaatcagccccagcaagaggaagttcagcatggagccaggtgacgaggacctagactgtgacaacgaccacgtctccaaaatgagtcgcatcttcaacccccatctgaacaagactgccaatggagactgccgcagagacccccgggagcggagccgcagccccatcgagcgcgctgtggcccccaccatgagcctgcacggcagccacctgtacacctccctccccagccttggcctggagcagcccctcgcactgaccaagaacagcctggacgccagcaggccagccggcctctcgcccacactgaccccgggggagcggcagcagaaccggccctccgtgatcacctgtgcctcggctggcgcccgcaactgcaacctctcgcactgccccatcgcgcacagcggctgtgccgcgcccgggcctgccagctaccggaggccaccgagcgctgccaccacctgtgaccccgtggtggaggaggcagcccgcaggagcctgggcaagaattacaaggagcccgagccggcacccaactccgtgtccatcacgggctccgtggacgacgcagctgccaaagctctgggtgacacgtggctccagatcaaagcggccaaggacggagcatccagcagccctgagtccgcctctcgcaggggccagcccgccagcccctctgcccacatggtcagccacagtcactccccctctgtggtctcc

SEQ ID NO: 23 - protein, 296 aa, synthetic construct
  REGION 1..296: note = Laboratory-synthesized seqeunce
MLFMKMDLLNYQYLDKMNNNIGILCYEGEAALRGEPRIQTLPVASALSSHRTGPPPISPSKRKFSMEPGDEDLDCDNDHVSKMSRIFNPHLNKTANGDCRRDPRERSRSPIERAVAPTMSLHGSHLYTSLPSLGLEQPLALTKNSLDASRPAGLSPTLTPGERQQNRPSVITCASAGARNCNLSHCPIAHSGCAAPGPASYRRPPSAATTCDPVVEEAARRSLGKNYKEPEPAPNSVSITGSVDDAAAKALGDTWLQIKAAKDGASSSPESASRRGQPASPSAHMVSHSHSPSVVS

SEQ ID NO: 24 - DNA, 870 bp, synthetic construct
  misc_feature 1..870: note = Laboratory-synthesized seqeunce
  source 1..870: mol_type = other DNA
atggagacgccattggatgttttgtccagggcagcatctctggtgcatgctgatgacgaaaaacgcgaagctgctctcaggggagaacccagaatacagaccctgccggtggcctctgccctcagcagtcaccgcaccggccctcccccaatcagccccagcaagaggaagttcagcatggagccaggtgacgaggacctagactgtgacaacgaccacgtctccaaaatgagtcgcatcttcaacccccatctgaacaagactgccaatggagactgccgcagagacccccgggagcggagccgcagccccatcgagcgcgctgtggcccccaccatgagcctgcacggcagccacctgtacacctccctccccagccttggcctggagcagcccctcgcactgaccaagaacagcctggacgccagcaggccagccggcctctcgcccacactgaccccgggggagcggcagcagaaccggccctccgtgatcacctgtgcctcggctggcgcccgcaactgcaacctctcgcactgccccatcgcgcacagcggctgtgccgcgcccgggcctgccagctaccggaggccaccgagcgctgccaccacctgtgaccccgtggtggaggaggcagcccgcaggagcctgggcaagaattacaaggagcccgagccggcacccaactccgtgtccatcacgggctccgtggacgacgcagctgccaaagctctgggtgacacgtggctccagatcaaagcggccaaggacggagcatccagcagccctgagtccgcctctcgcaggggccagcccgccagcccctctgcccacatggtcagccacagtcactccccctctgtggtctcc

SEQ ID NO: 25 - protein, 290 aa, synthetic construct
  REGION 1..290: note = Laboratory-synthesized seqeunce
METPLDVLSRAASLVHADDEKREAALRGEPRIQTLPVASALSSHRTGPPPISPSKRKFSMEPGDEDLDCDNDHVSKMSRIFNPHLNKTANGDCRRDPRERSRSPIERAVAPTMSLHGSHLYTSLPSLGLEQPLALTKNSLDASRPAGLSPTLTPGERQQNRPSVITCASAGARNCNLSHCPIAHSGCAAPGPASYRRPPSAATTCDPVVEEAARRSLGKNYKEPEPAPNSVSITGSVDDAAAKALGDTWLQIKAAKDGASSSPESASRRGQPASPSAHMVSHSHSPSVVS

SEQ ID NO: 26 - DNA, 630 bp, synthetic construct
  misc_feature 1..630: note = Laboratory-synthesized seqeunce
  source 1..630: mol_type = other DNA
atgattaaagtgaggaacaagactgccaatggagactgccgcagagacccccgggagcggagccgcagccccatcgagcgcgctgtggcccccaccatgagcctgcacggcagccacctgtacacctccctccccagccttggcctggagcagcccctcgcactgaccaagaacagcctggacgccagcaggccagccggcctctcgcccacactgaccccgggggagcggcagcagaaccggccctccgtgatcacctgtgcctcggctggcgcccgcaactgcaacctctcgcactgccccatcgcgcacagcggctgtgccgcgcccgggcctgccagctaccggaggccaccgagcgctgccaccacctgtgaccccgtggtggaggaggcagcccgcaggagcctgggcaagaattacaaggagcccgagccggcacccaactccgtgtccatcacgggctccgtggacgacgcagctgccaaagctctgggtgacacgtggctccagatcaaagcggccaaggacggagcatccagcagccctgagtccgcctctcgcaggggccagcccgccagcccctctgcccacatggtcagccacagtcactccccctctgtggtctcc

SEQ ID NO: 27 - protein, 210 aa, synthetic construct
  REGION 1..210: note = Laboratory-synthesized seqeunce
MIKVRNKTANGDCRRDPRERSRSPIERAVAPTMSLHGSHLYTSLPSLGLEQPLALTKNSLDASRPAGLSPTLTPGERQQNRPSVITCASAGARNCNLSHCPIAHSGCAAPGPASYRRPPSAATTCDPVVEEAARRSLGKNYKEPEPAPNSVSITGSVDDAAAKALGDTWLQIKAAKDGASSSPESASRRGQPASPSAHMVSHSHSPSVVS

SEQ ID NO: 28 - DNA, 618 bp, synthetic construct
  misc_feature 1..618: note = Laboratory-synthesized seqeunce
  source 1..618: mol_type = other DNA
atgaacaagactgccaatggagactgccgcagagacccccgggagcggagccgcagccccatcgagcgcgctgtggcccccaccatgagcctgcacggcagccacctgtacacctccctccccagccttggcctggagcagcccctcgcactgaccaagaacagcctggacgccagcaggccagccggcctctcgcccacactgaccccgggggagcggcagcagaaccggccctccgtgatcacctgtgcctcggctggcgcccgcaactgcaacctctcgcactgccccatcgcgcacagcggctgtgccgcgcccgggcctgccagctaccggaggccaccgagcgctgccaccacctgtgaccccgtggtggaggaggcagcccgcaggagcctgggcaagaattacaaggagcccgagccggcacccaactccgtgtccatcacgggctccgtggacgacgcagctgccaaagctctgggtgacacgtggctccagatcaaagcggccaaggacggagcatccagcagccctgagtccgcctctcgcaggggccagcccgccagcccctctgcccacatggtcagccacagtcactccccctctgtggtctcc

SEQ ID NO: 29 - protein, 206 aa, synthetic construct
  REGION 1..206: note = Laboratory-synthesized seqeunce
MNKTANGDCRRDPRERSRSPIERAVAPTMSLHGSHLYTSLPSLGLEQPLALTKNSLDASRPAGLSPTLTPGERQQNRPSVITCASAGARNCNLSHCPIAHSGCAAPGPASYRRPPSAATTCDPVVEEAARRSLGKNYKEPEPAPNSVSITGSVDDAAAKALGDTWLQIKAAKDGASSSPESASRRGQPASPSAHMVSHSHSPSVVS

SEQ ID NO: 30 - DNA, 885 bp, synthetic construct
  misc_feature 1..885: note = Laboratory-synthesized seqeunce
  source 1..885: mol_type = other DNA
atgactgagaatacgcattttgacaaaatccctgagtcctgtgcactcaaaagttggagacatccaggtctgcaccatggcgaagctgctctcaggggagaacccagaatacagaccctgccggtggcctctgccctcagcagtcaccgcaccggccctcccccaatcagccccagcaagaggaagttcagcatggagccaggtgacgaggacctagactgtgacaacgaccacgtctccaaaatgagtcgcatcttcaacccccatctgaacaagactgccaatggagactgccgcagagacccccgggagcggagccgcagccccatcgagcgcgctgtggcccccaccatgagcctgcacggcagccacctgtacacctccctccccagccttggcctggagcagcccctcgcactgaccaagaacagcctggacgccagcaggccagccggcctctcgcccacactgaccccgggggagcggcagcagaaccggccctccgtgatcacctgtgcctcggctggcgcccgcaactgcaacctctcgcactgccccatcgcgcacagcggctgtgccgcgcccgggcctgccagctaccggaggccaccgagcgctgccaccacctgtgaccccgtggtggaggaggcagcccgcaggagcctgggcaagaattacaaggagcccgagccggcacccaactccgtgtccatcacgggctccgtggacgacgcagctgccaaagctctgggtgacacgtggctccagatcaaagcggccaaggacggagcatccagcagccctgagtccgcctctcgcaggggccagcccgccagcccctctgcccacatggtcagccacagtcactccccctctgtggtctcc

SEQ ID NO: 31 - protein, 295 aa, synthetic construct
  REGION 1..295: note = Laboratory-synthesized seqeunce
MTENTHFDKIPESCALKSWRHPGLHHGEAALRGEPRIQTLPVASALSSHRTGPPPISPSKRKFSMEPGDEDLDCDNDHVSKMSRIFNPHLNKTANGDCRRDPRERSRSPIERAVAPTMSLHGSHLYTSLPSLGLEQPLALTKNSLDASRPAGLSPTLTPGERQQNRPSVITCASAGARNCNLSHCPIAHSGCAAPGPASYRRPPSAATTCDPVVEEAARRSLGKNYKEPEPAPNSVSITGSVDDAAAKALGDTWLQIKAAKDGASSSPESASRRGQPASPSAHMVSHSHSPSVVS

SEQ ID NO: 32 - DNA, 693 bp, synthetic construct
  misc_feature 1..693: note = Laboratory-synthesized seqeunce
  source 1..693: mol_type = other DNA
atggagccaggtgacgaggacctagactgtgacaacgaccacgtctccaaaatgagtcgcatcttcaacccccatctgaacaagactgccaatggagactgccgcagagacccccgggagcggagccgcagccccatcgagcgcgctgtggcccccaccatgagcctgcacggcagccacctgtacacctccctccccagccttggcctggagcagcccctcgcactgaccaagaacagcctggacgccagcaggccagccggcctctcgcccacactgaccccgggggagcggcagcagaaccggccctccgtgatcacctgtgcctcggctggcgcccgcaactgcaacctctcgcactgccccatcgcgcacagcggctgtgccgcgcccgggcctgccagctaccggaggccaccgagcgctgccaccacctgtgaccccgtggtggaggaggcagcccgcaggagcctgggcaagaattacaaggagcccgagccggcacccaactccgtgtccatcacgggctccgtggacgacgcagctgccaaagctctgggtgacacgtggctccagatcaaagcggccaaggacggagcatccagcagccctgagtccgcctctcgcaggggccagcccgccagcccctctgcccacatggtcagccacagtcactccccctctgtggtctcc

SEQ ID NO: 33 - protein, 231 aa, synthetic construct
  REGION 1..231: note = Laboratory-synthesized seqeunce
MEPGDEDLDCDNDHVSKMSRIFNPHLNKTANGDCRRDPRERSRSPIERAVAPTMSLHGSHLYTSLPSLGLEQPLALTKNSLDASRPAGLSPTLTPGERQQNRPSVITCASAGARNCNLSHCPIAHSGCAAPGPASYRRPPSAATTCDPVVEEAARRSLGKNYKEPEPAPNSVSITGSVDDAAAKALGDTWLQIKAAKDGASSSPESASRRGQPASPSAHMVSHSHSPSVVS

SEQ ID NO: 34 - DNA, 133 bp, synthetic construct
  misc_feature 1..133: note = Laboratory-synthesized seqeunce
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 35 - DNA, 2096 bp, synthetic construct
  misc_feature 1..2096: note = Laboratory-synthesized seqeunce
  source 1..2096: mol_type = other DNA
gcatgccaatttatagtgccgtcactaacagtactgatactttaacatgctaagtttaaagtgtgtgctatattaattgtaagattggtgaagagaggtgttatcagatggaagctgcacatttctggattaatgtggttaaatgtatcttctcctgtgattactgtctttatttcttcttttaaaatattgtcatttggacatctatctgtatagctacgccctgacacgtcctcctggagacagataagaagttacgacgggaggagcagatggaggcaaagcgctgtgatgcttttgtggtttgagtgcacacatttgttcagtgattctgtgaaatgagtgagcaaatggtgaccgggtgccctgtaaatggtgttctacatcttaagagaagaacacggacactaggtaagtgaagcttgctgtcactcctctacagcgtcacagagggtcagtcacccttgaccacactgaactagtcgtcacctttccactcttcctgccagaagagcagaaatcagactctctggggatatcagcctcacccctactgctctctccattatgaggcaaactttctttcacttcccagaggctctgggggcagcaaggtcaaccctttcctcagactctagtctcggaggagatcagatcgcgcttattcaagggaaccagcccctgctctgcgccctggtccaaggctgttgaagagtgacaaaaggcaccacgctgcggggacgcgggtgaagcccctctgtgtgtcctctgggcataatcaggaactggtgccaaatcagaggtgatgtggccagggctttgggagtgacgcgcggctgggaggcttgcgcacccaaggcacgcccctgccaagtcccactagcagctctttggagacctgggccggctcagccacttcccccagtccctcctccggcaaggggctatatagatctcccaggtcagggcgcagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcatgctatttatgaagatggacctgttgaactatcagtacttggacaagatgaacaacaatatcggcattctgtgctacgaaggcgaagctgctctcaggggagaacccagaatgcagaccctgccggtggcctctgccctcagcagtcaccgcaccggccctcccccaatcagccccagcaagaggaagttcagcatggagccaggtgacgaggacctagactgtgacaacgaccacgtctccaaaatgagtcgcatcttcaacccccatctgaacaagactgccaatggagactgccgcagagacccccgggagcggagccgcagccccatcgagcgcgctgtggcccccaccatgagcctgcacggcagccacctgtacacctccctccccagccttggcctggagcagcccctcgcactgaccaagaacagcctggacgccagcaggccagccggcctctcgcccacactgaccccgggggagcggcagcagaaccggccctccgtgatcacctgtgcctcggctggcgcccgcaactgcaacctctcgcactgccccatcgcgcacagcggctgtgccgcgcccgggcctgccagctaccggaggccaccgagcgctgccaccacctgtgaccccgtggtggaggagcatttccgcaggagcctgggcaagaattacaaggagcccgagccggcacccaactccgtgtccatcacgggctccgtggacgaccactttgccaaagctctgggtgacacgtggctccagatcaaagcggccaaggacggagcatccagcagccctgagtccgcctctcgcaggggccagcccgccagcccctctgcccacatggtcagccacagtcactccccctctgtggtctccgcc

SEQ ID NO: 36 - DNA, 2096 bp, synthetic construct
  misc_feature 1..2096: note = Laboratory-synthesized seqeunce
  source 1..2096: mol_type = other DNA
gcatgccaatttatagtgccgtcactaacagtactgatactttaacatgctaagtttaaagtgtgtgctatattaattgtaagattggtgaagagaggtgttatcagatggaagctgcagcagcctggattaatgtggttaaatgtatcttctcctgtgattactgtctttatttcttcttttaaaatattgtcatttggacatctatctgtatagctacgccctgacacgtcctcctggagacagataagaagttacgacgggaggagcagatggaggcaaagcgctgtgatgcttttgtggtttgagtgcacacatttgttcagtgattctgtgaaatgagtgagcaaatggtgaccgggtgccctgtaaatggtgttctacatcttaagagaagaacacggacactaggtaagtgaagcttgctgtcactcctctacagcgtcacagagggtcagtcacccttgaccacactgaactagtcgtcacctttccactcttcctgccagaagagcagaaatcagactctctggggatatcagcctcacccctactgctctctccattatgaggcaaactttctttcacttcccagaggctctgggggcagcaaggtcaaccctttcctcagactctagtctcggaggagatcagatcgcgcttattcaagggaaccagcccctgctctgcgccctggtccaaggctgttgaagagtgacaaaaggcaccacgctgcggggacgcgggtgaagcccctctgtgtgtcctctgggcataatcaggaactggtgccaaatcagaggtgatgtggccagggctttgggagtgacgcgcggctgggaggcttgcgcacccaaggcacgcccctgccaagtcccactagcagctctttggagacctgggccggctcagccacttcccccagtccctcctccggcaaggggctatatagatctcccaggtcagggcgcagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatcgcagctgcctttctctccacaggtgtccactcccagttcaattacagctcttaaggctagagtacttaatacgactcactataggctagcatgctatttatgaagatggacctgttgaactatcagtacttggacaagatgaacaacaatatcggcattctgtgctacgaaggcgaagctgctctcaggggagaacccagaatgcagaccctgccggtggcctctgccctcagcagtcaccgcaccggccctcccccaatcagccccagcaagaggaagttcagcatggagccaggtgacgaggacctagactgtgacaacgaccacgtctccaaaatgagtcgcatcttcaacccccatctgaacaagactgccaatggagactgccgcagagacccccgggagcggagccgcagccccatcgagcgcgctgtggcccccaccatgagcctgcacggcagccacctgtacacctccctccccagccttggcctggagcagcccctcgcactgaccaagaacagcctggacgccagcaggccagccggcctctcgcccacactgaccccgggggagcggcagcagaaccggccctccgtgatcacctgtgcctcggctggcgcccgcaactgcaacctctcgcactgccccatcgcgcacagcggctgtgccgcgcccgggcctgccagctaccggaggccaccgagcgctgccaccacctgtgaccccgtggtggaggaggcagcccgcaggagcctgggcaagaattacaaggagcccgagccggcacccaactccgtgtccatcacgggctccgtggacgacgcagctgccaaagctctgggtgacacgtggctccagatcaaagcggccaaggacggagcatccagcagccctgagtccgcctctcgcaggggccagcccgccagcccctctgcccacatggtcagccacagtcactccccctctgtggtctccgcc

SEQ ID NO: 37 - protein, 239 aa, synthetic construct
  REGION 1..239: note = Laboratory-synthesized seqeunce
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 38 - DNA, 720 bp, synthetic construct
  misc_feature 1..720: note = Laboratory-synthesized seqeunce
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 39 - DNA, 2816 bp, synthetic construct
  misc_feature 1..2816: note = Laboratory-synthesized seqeunce
  source 1..2816: mol_type = other DNA
gcatgccaatttatagtgccgtcactaacagtactgatactttaacatgctaagtttaaagtgtgtgctatattaattgtaagattggtgaagagaggtgttatcagatggaagctgcacatttctggattaatgtggttaaatgtatcttctcctgtgattactgtctttatttcttcttttaaaatattgtcatttggacatctatctgtatagctacgccctgacacgtcctcctggagacagataagaagttacgacgggaggagcagatggaggcaaagcgctgtgatgcttttgtggtttgagtgcacacatttgttcagtgattctgtgaaatgagtgagcaaatggtgaccgggtgccctgtaaatggtgttctacatcttaagagaagaacacggacactaggtaagtgaagcttgctgtcactcctctacagcgtcacagagggtcagtcacccttgaccacactgaactagtcgtcacctttccactcttcctgccagaagagcagaaatcagactctctggggatatcagcctcacccctactgctctctccattatgaggcaaactttctttcacttcccagaggctctgggggcagcaaggtcaaccctttcctcagactctagtctcggaggagatcagatcgcgcttattcaagggaaccagcccctgctctgcgccctggtccaaggctgttgaagagtgacaaaaggcaccacgctgcggggacgcgggtgaagcccctctgtgtgtcctctgggcataatcaggaactggtgccaaatcagaggtgatgtggccagggctttgggagtgacgcgcggctgggaggcttgcgcacccaaggcacgcccctgccaagtcccactagcagctctttggagacctgggccggctcagccacttcccccagtccctcctccggcaaggggctatatagatctcccaggtcagggcgcagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcatgctatttatgaagatggacctgttgaactatcagtacttggacaagatgaacaacaatatcggcattctgtgctacgaaggcgaagctgctctcaggggagaacccagaatgcagaccctgccggtggcctctgccctcagcagtcaccgcaccggccctcccccaatcagccccagcaagaggaagttcagcatggagccaggtgacgaggacctagactgtgacaacgaccacgtctccaaaatgagtcgcatcttcaacccccatctgaacaagactgccaatggagactgccgcagagacccccgggagcggagccgcagccccatcgagcgcgctgtggcccccaccatgagcctgcacggcagccacctgtacacctccctccccagccttggcctggagcagcccctcgcactgaccaagaacagcctggacgccagcaggccagccggcctctcgcccacactgaccccgggggagcggcagcagaaccggccctccgtgatcacctgtgcctcggctggcgcccgcaactgcaacctctcgcactgccccatcgcgcacagcggctgtgccgcgcccgggcctgccagctaccggaggccaccgagcgctgccaccacctgtgaccccgtggtggaggagcatttccgcaggagcctgggcaagaattacaaggagcccgagccggcacccaactccgtgtccatcacgggctccgtggacgaccactttgccaaagctctgggtgacacgtggctccagatcaaagcggccaaggacggagcatccagcagccctgagtccgcctctcgcaggggccagcccgccagcccctctgcccacatggtcagccacagtcactccccctctgtggtctccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 40 - DNA, 2816 bp, synthetic construct
  misc_feature 1..2816: note = Laboratory-synthesized seqeunce
  source 1..2816: mol_type = other DNA
gcatgccaatttatagtgccgtcactaacagtactgatactttaacatgctaagtttaaagtgtgtgctatattaattgtaagattggtgaagagaggtgttatcagatggaagctgcagcagcctggattaatgtggttaaatgtatcttctcctgtgattactgtctttatttcttcttttaaaatattgtcatttggacatctatctgtatagctacgccctgacacgtcctcctggagacagataagaagttacgacgggaggagcagatggaggcaaagcgctgtgatgcttttgtggtttgagtgcacacatttgttcagtgattctgtgaaatgagtgagcaaatggtgaccgggtgccctgtaaatggtgttctacatcttaagagaagaacacggacactaggtaagtgaagcttgctgtcactcctctacagcgtcacagagggtcagtcacccttgaccacactgaactagtcgtcacctttccactcttcctgccagaagagcagaaatcagactctctggggatatcagcctcacccctactgctctctccattatgaggcaaactttctttcacttcccagaggctctgggggcagcaaggtcaaccctttcctcagactctagtctcggaggagatcagatcgcgcttattcaagggaaccagcccctgctctgcgccctggtccaaggctgttgaagagtgacaaaaggcaccacgctgcggggacgcgggtgaagcccctctgtgtgtcctctgggcataatcaggaactggtgccaaatcagaggtgatgtggccagggctttgggagtgacgcgcggctgggaggcttgcgcacccaaggcacgcccctgccaagtcccactagcagctctttggagacctgggccggctcagccacttcccccagtccctcctccggcaaggggctatatagatctcccaggtcagggcgcagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatcgcagctgcctttctctccacaggtgtccactcccagttcaattacagctcttaaggctagagtacttaatacgactcactataggctagcatgctatttatgaagatggacctgttgaactatcagtacttggacaagatgaacaacaatatcggcattctgtgctacgaaggcgaagctgctctcaggggagaacccagaatgcagaccctgccggtggcctctgccctcagcagtcaccgcaccggccctcccccaatcagccccagcaagaggaagttcagcatggagccaggtgacgaggacctagactgtgacaacgaccacgtctccaaaatgagtcgcatcttcaacccccatctgaacaagactgccaatggagactgccgcagagacccccgggagcggagccgcagccccatcgagcgcgctgtggcccccaccatgagcctgcacggcagccacctgtacacctccctccccagccttggcctggagcagcccctcgcactgaccaagaacagcctggacgccagcaggccagccggcctctcgcccacactgaccccgggggagcggcagcagaaccggccctccgtgatcacctgtgcctcggctggcgcccgcaactgcaacctctcgcactgccccatcgcgcacagcggctgtgccgcgcccgggcctgccagctaccggaggccaccgagcgctgccaccacctgtgaccccgtggtggaggaggcagcccgcaggagcctgggcaagaattacaaggagcccgagccggcacccaactccgtgtccatcacgggctccgtggacgacgcagctgccaaagctctgggtgacacgtggctccagatcaaagcggccaaggacggagcatccagcagccctgagtccgcctctcgcaggggccagcccgccagcccctctgcccacatggtcagccacagtcactccccctctgtggtctccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 41 - protein, 16 aa, Homo sapiens
DPVVEEHFRRSLGKNY

SEQ ID NO: 42 - protein, 16 aa, Homo sapiens
TGSVDDHFAKALGDTW

SEQ ID NO: 43 - protein, 16 aa, synthetic construct
  REGION 1..16: note = Homo sapiens
DPVVEEAARRSLGKNY

SEQ ID NO: 44 - protein, 16 aa, synthetic construct
  REGION 1..16: note = Homo sapiens
TGSVDDAAAKALGDTW

(19) United States
(12) Patent Application Publication
(10) Pub. No.: US 2025/0333457 A1
(43) Pub. Date: Oct. 30, 2025

(54) VGLL4 WITH UCP-1 CIS-REGULATORY ELEMENT AND METHOD OF USE THEREOF

(71) Applicant: MASONIC MEDICAL RESEARCH LABORATORY, Utica, NY (US)

(72) Inventor: Zhiqiang LIN, Utica, NY (US)

(73) Assignee: MASONIC MEDICAL RESEARCH LABORATORY, Utica, NY (US)

(21) Appl. No.: 19/264,969

(22) Filed: Jul. 10, 2025

Related U.S. Application Data
(63) Continuation of application No. 17/731,201, filed on Apr. 27, 2022, now Pat. No. 12,384,823.
Application No. 17/731,201 is a continuation of application No. 16/925,632, filed on Jul. 10, 2020, now Pat. No. 11,319,354.
(60) Provisional application No. 62/872,624, filed on Jul. 10, 2019.

(51) Int. Cl. C07K 14/47; C12N 15/86

(52) U.S. Cl. CPC C07K 14/4705; C12N 15/86; C12N 2750/14143; C12N 2830/001

(57) ABSTRACT

Provided is a polynucleotide, including a cis-regulatory element and a nucleotide sequence encoding a vestigial like 4 protein, wherein the cis-regulatory element includes an uncoupling protein 1 enhancer and an uncoupling protein 1 promoter. Also provided is a viral vector including said polynucleotide. Also provided is a method of transfecting a cell or a subject with said polynucleotide or said viral vector.

### CROSS-REFERENCE TO RELATED APPLICATIONS

This application is a Non-Provisional U.S. Patent Application and claims priority to U.S. Provisional Application continuation of U.S. Patent Application No. 62/872,624, filed Jul. 10, 2019, the entire contents of which is incorporated herewith in its entirety.

### GOVERNMENT RIGHTS STATEMENT

This invention was made with Government support under grant number HL138454 awarded by the National Institutes of Health. The Government has certain rights in the invention.

### SEQUENCE LISTING

The instant application contains a Sequence Listing, created on Jul. 9, 2020; the file, in ASCII format, is designated H1789760.txt and is 65.9 KB in size. The file is hereby incorporated by reference in its entirety into the instant application.

### BACKGROUND

Obesity is a global epidemic that plagues the human society, threatening the health of both adult and children. Effective pharmacological therapies for obesity are urgently needed. Obesity-related pathologies include, among others, diabetes and liver disease. Adipose tissue overgrowth is the root of obesity, with deleterious health effects. Adipose tissue is composed of white and brown adipose tissue (BAT). White adipose tissue (WAT) stores triglycerides in adipocytes, and BAT burns triglycerides and glucose for generating heat. The development of obesity depends not only on the balance between food intake and caloric utilization but also on the balance between BAT and WAT. Higher BAT is correlated with leanness in the adult and greater muscle volume in children, indicating that functional BAT benefits both energy homeostasis and muscle growth. In humans, BAT is abundant in infants, and decreases with age. Recently, the discovery of functional BAT in adult individuals raised the possibility of treating obesity by activating BAT. However, compositions and methods for increasing BAT are lacking. The present disclosure is directed to overcoming these and other deficiencies in the art.

### SUMMARY

The following disclosure includes improvements over such shortcomings.

In an aspect, provided is a polynucleotide, including a cis-regulatory element and a nucleotide sequence encoding a vestigial like 4 protein, wherein the cis-regulatory element includes an uncoupling protein 1 enhancer and an uncoupling protein 1 promoter. In an example, the uncoupling protein 1 enhancer has at least 90% identity with a sequence selected from SEQ ID NO: 1, SEQ ID NO 4, and SEQ ID NO: 7. In another example, the uncoupling protein 1 enhancer is selected from SEQ ID NO: 1, SEQ ID NO 4, and SEQ ID NO: 7. In another example, the uncoupling protein 1 promotor has at least 90% identity with a sequence selected from SEQ ID NO: 2, SEQ ID NO 5, and SEQ ID NO: 8. In still another example, the uncoupling protein 1 promotor is selected from SEQ ID NO: 2, SEQ ID NO 5, and SEQ ID NO: 8. In yet another example, the cis-regulatory element has at least 90% homology with a sequence selected from SEQ ID NO: 3, SEQ ID NO: 6, and SEQ ID NO: 9. In a further example, the cis-regulatory element is selected from SEQ ID NO: 3, SEQ ID NO: 6, and SEQ ID NO: 9.

In another example, the vestigial like 4 protein has at least 90% homology with a sequence selected from SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, and SEQ ID NO: 33. In still another example, the vestigial like 4 protein is selected from SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, and SEQ ID NO: 33. In yet another example, the sequence encoding a vestigial like 4 protein has at least 90% identity with a sequence selected from SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, and SEQ ID NO: 32. In a further example, the sequence encoding a vestigial like 4 protein is selected from SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, and SEQ ID NO: 32.

In another example, the vestigial like 4 protein has from 0 to 3 substitutions to a sequence selected from SEQ ID NO: 11, SEQ ID NO: 13, SEQ ID NO: 15, SEQ ID NO: 17, SEQ ID NO: 19, and SEQ ID NO: 21, wherein the substitutions are not in a TDU domain. In still another example, the vestigial like 4 protein has from 0 to 3 substitutions to a sequence selected from SEQ ID NO: 23, SEQ ID NO: 25, SEQ ID NO: 27, SEQ ID NO: 29, SEQ ID NO: 31, and SEQ ID NO: 33, wherein the substitutions are not in a TDU domain.

Another example further includes an intron between the cis-regulatory element and the nucleotide sequence encoding a vestigial like 4 protein. In another example, the intron has at least 90% homology with SEQ ID NO: 34. In still another example, the intron is SEQ ID NO: 34.

Another example includes a nucleotide sequence having at least 90% homology with SEQ ID NO: 35. An example includes a nucleotide sequence of SEQ ID NO: 35. Another example includes a nucleotide sequence having at least 90% homology with SEQ ID NO: 36. An example includes a nucleotide sequence of SEQ ID NO: 36.

Another example further includes a nucleotide sequence encoding a reporter protein. In another example, the reporter protein is selected from a green fluorescent protein, a yellow fluorescent protein, a red fluorescent protein, a blue fluorescent protein, a luciferase protein, a beta-galactosidase protein, a glutathione S-transferase protein, a chloramphenicol acetyltransferase protein, and any combination of two or more of the foregoing. In still another example, the reporter protein includes a green fluorescent protein. In yet another example, the reporter protein includes SEQ ID NO: 37. In a further example, the nucleotide sequence encoding a reporter protein includes SEQ ID NO: 38.

Another example includes a nucleotide sequence having at least 90% homology with SEQ ID NO: 39. An example includes SEQ ID NO: 39. Another example includes a nucleotide sequence having at least 90% homology with SEQ ID NO: 40. An example includes SEQ ID NO: 40.

In another aspect, provided is a viral vector including any of the foregoing examples of a polynucleotide that include a cis-regulatory element and a nucleotide sequence encoding a vestigial like 4 protein. In an example, the viral vector includes an adenoviral associated vector.

In another aspect, provided is a cell transfected with any of the foregoing examples of a polynucleotide that include a cis-regulatory element and a nucleotide sequence encoding a vestigial like 4 protein. In an example, the cell was contacted with any of the foregoing examples of a viral vector that include any of the foregoing examples of a polynucleotide that include a cis-regulatory element and a nucleotide sequence encoding a vestigial like 4 protein.

In still another aspect, provided is an organism transfected with any of the foregoing examples of a polynucleotide that include a cis-regulatory element and a nucleotide sequence encoding a vestigial like 4 protein. In an example, the organism was contacted with any of the foregoing examples of a viral vector.

In another aspect, provided is a method. In an example, the method includes transfecting a cell with any of the foregoing examples of a polynucleotide that include a cis-regulatory element and a nucleotide sequence encoding a vestigial like 4 protein. In another example, transfecting includes contacting the cell with any of the foregoing examples of a viral vector that include any of the foregoing examples of a polynucleotide that include a cis-regulatory element and a nucleotide sequence encoding a vestigial like 4 protein. In still another example includes transfecting an organism with any of the foregoing examples of a polynucleotide that include a cis-regulatory element and a nucleotide sequence encoding a vestigial like 4 protein. In yet another example, transfecting includes contacting the organism with any of the foregoing examples of a viral vector that include any of the foregoing examples of a polynucleotide that include a cis-regulatory element and a nucleotide sequence encoding a vestigial like 4 protein.

In another example, the organism is a mammal. In still another example, the organism is a human.

In an example of the method, the vestigial like protein 4 does not include an HF to AA substitution in a TDU domain of the vestigial like protein 4 and the transfecting includes increasing a ratio of a volume of brown adipose tissue to a volume of white adipose tissue in the organism. In still another example, the vestigial like protein 4 does not include an HF to AA substitution in a TDU domain of the vestigial like protein 4 and the transfecting includes increasing a volume of brown adipose tissue in the organism, decreasing the volume of white adipose tissue in the organism, or both. In yet another example, the vestigial like protein 4 does not comprise an HF to AA substitution in a TDU domain and the transfecting includes reducing a ratio of a volume of adipose tissue to a volume of non-adipose tissue in the organism.

In a further example, the vestigial like protein 4 does not include an HF to AA substitution in a TDU domain, and the organism is obese or is at risk of developing obesity. In still a further example, the vestigial like protein 4 does not include an HF to AA substitution in a TDU domain, and the transfecting includes preventing obesity in the organism. In yet another example, the vestigial like protein 4 does not include an HF to AA substitution in a TDU domain, and the transfecting includes treating obesity in the organism. In another example, the vestigial like protein 4 does not include an HF to AA substitution in a TDU domain, and the transfecting includes reducing obesity in the organism.

In an example of the method, the vestigial like protein 4 includes an HF to AA substitution in each of two TDU domains, wherein the transfecting includes reducing a volume of adipose tissue of the organism. In another example, the vestigial like protein 4 includes an HF to AA substitution in each of two TDU domains, and the transfecting includes reducing a volume of brown adipose tissue of the organism. In still another example, the vestigial like protein 4 includes an HF to AA substitution in a TDU domain, and the organism is obese or is at risk of developing obesity. In yet another example, the vestigial like protein 4 includes an HF to AA substitution in a TDU domain, and the transfecting includes preventing obesity in the organism. In a further example, the transfecting includes treating obesity in the organism. In still a further example, the vestigial like protein 4 includes an HF to AA substitution in a TDU domain, and the transfecting includes reducing obesity in the organism.

In an example of the method, the vestigial like protein 4 includes an HF to AA substitution in each of two TDU domains, and the transfecting includes reducing fatty acid synthesis in the organism. In another example, the vestigial like protein 4 includes an HF to AA substitution in a TDU domain, and the organism has hepatic steatosis or is at risk for developing hepatic steatosis. In still another example, the vestigial like protein 4 includes an HF to AA substitution in each of two TDU domains, and the transfecting includes preventing hepatic steatosis in the organism. In yet another example, the vestigial like protein 4 includes an HF to AA substitution in each of two TDU domains, and the transfecting includes treating hepatic steatosis in the organism. In a further example, the vestigial like protein 4 includes an HF to AA substitution in each of two TDU domains, and the transfecting includes reducing hepatic steatosis in the organism.

In an example of the method, the vestigial like protein 4 includes an HF to AA substitution in each of two TDU domains, and the organism has diabetes or is at risk of developing diabetes. In another example, the vestigial like protein 4 includes an HF to AA substitution in each of two TDU domains, wherein the transfecting includes preventing diabetes in the organism. In still another example, the vestigial like protein 4 includes an HF to AA substitution in each of two TDU domains, wherein the transfecting includes treating diabetes in the organism.

### BRIEF DESCRIPTION OF THE DRAWINGS

These and other features, aspects, and advantages of the present disclosure will become better understood when the following detailed description is read with reference to the accompanying drawings, wherein:

FIG. 1 shows a schematic view of constructs in accordance with aspects of the present disclosure.

FIG. 2 shows timing of vector administration and expression measurement in accordance with aspects of the present disclosure.

FIG. 3 shows representative bioluminescence images of a dorsal view of mice administered a viral vector in accordance with aspects of the present disclosure.

FIG. 4 shows representative bioluminescence images of a ventral view of mice administered a viral vector in accordance with aspects of the present disclosure.

FIG. 5 shows bioluminescence signal origin following viral vector administration in accordance with aspects of the present disclosure.

FIG. 6 shows immunofluorescence staining images of interscapular BAT following viral vector administration in accordance with aspects of the present disclosure.

FIG. 7 shows immunofluorescence staining images of interscapular BAT and WAT following viral vector administration in accordance with aspects of the present disclosure.

FIG. 8 shows a schematic view of a construct in accordance with aspects of the present disclosure.

FIG. 9 shows timing of vector administration and BAT and WAT measurement in accordance with aspects of the present disclosure.

FIG. 10 shows relative BAT and WAT expression following treatment with control viral vector in accordance with aspects of the present disclosure.

FIG. 11 shows relative BAT and WAT expression following treatment with a viral vector including a UCP-1 cis-regulatory element and Vgl14 coding sequence in accordance with aspects of the present disclosure.

FIG. 12 shows an effect of treatment with a viral vector including a UCP-1 cis-regulatory element and Vgl14 coding sequence on BAT volume in accordance with aspects of the present disclosure.

FIG. 13 shows an effect of treatment with a viral vector including a UCP-1 cis-regulatory element and Vgll4 coding sequence on WAT volume in accordance with aspects of the present disclosure.

FIG. 14 shows an effect of treatment with a viral vector including a UCP-1 cis-regulatory element and Vgl14 coding sequence on the ratio of WAT volume to BAT volume in accordance with aspects of the present disclosure.

FIG. 15 shows bioluminescent expression of a reporter protein, luciferase, in 6-week old mice administered viral vectors including cis-regulatory elements in accordance with the present disclosure.

FIG. 16 shows quantification of luciferase activity in brown adipose tissue (BAT).

FIG. 17 shows quantification of luciferase activity in liver.

FIG. 18 shows quantification of viral vector genome copies in BAT and liver as assessed by real-time PCR.

FIG. 19 is an illustration of the Hippo/YAP signaling cascade and Vgll4's inhibitory role in YAP/TEAD interactions.

FIG. 20 shows the mutations made to two TONDU (TDU_1 and TDU_2) domains of Vgll4 isoforms. Vgll4 isoforms A through F include two TDU domains, TDU_1 and TDU_2, with the indicated sequences (SEQ ID NO: 41 and SEQ ID NO: 42). The Vgll4-HF4A mutants of Vgll4 isoforms disclosed herein include two alanine substitutions to four amino acids, two in TDU_1 (a histidine to alanine and a phenylalanine to alanine) and two in TDU_2 (a histidine to alanine and a phenylalanine to alanine). Vgll4-HF4A have the dual-substituted TDU domains SEQ ID NO: 43 and SEQ NO ID: 44 (instead of SEQ ID NO: 41 and SEQ ID NO: 42, respectively).

FIG. 21 is an illustration showing that whereas Vgll4 disrupts a YAP/TEAD1 complex, Vgll4-HF4A mutants do not.

FIG. 22 Shows a schematic illustration of generation of brown adipocyte specific Tead1 knockout mice.

FIG. 23 is a western blot showing confirmation of depleted expression of TEAD1 in brown adipose tissue of the conditional TEAD1 knockout mice, with GADPH as a control.

FIG. 24 shows whole mount view of interscapular brown adipose tissue (iBAT) collected from 1-month old male mice. Bar=5 mm.

FIG. 25 shows the ration between iBAT and body weight ratio (*P<0.05).

FIG. 26 shows qRT-PCR measurement of various mRNA transcript levels in BAT, normalized to 36B4.

FIG. 27 is a genomic view of TEAD1 binding site in the Fgf21 promoter region.

FIG. 28 is an illustration of experimental design for injecting adult mice fed a high-fat diet with a viral vector in accordance with the present disclosure. GTT is glucose tolerance test and ITT is insulin tolerance test.

FIG. 29 shows bioluminescence imaging mice 8 weeks after infusion with viral vectors in accordance with the present disclosure.

FIG. 30 and FIG. 31 show total body weight measurements and accumulated body weight gain measurements of transfected mice, respectively.

FIGS. 32A, 32B, AND 32C show effects for transfection with a viral vector carrying a BCE-Vggl4 polynucleotide transcript on a ratio of total adipose tissue to tibia length, a ratio of total lean mass to tibia length, and a ratio of fat mass to lean mass. In 32A and 32B, AAV.Luciferase control is on the right and AAV. Vgll4 is on the right.

FIG. 33A shows an illustration of an experimental design demonstrating that pre-treatment of mice with AAV.BCE.VGLL4 mitigates body weight gain. AAV.BCE. Vgll4 was subcutaneously injected into the interscapular region of 8-weeks-old C57/BL6 mice. After 13 weeks high fat diet treatment, mice were tested for glucose tolerance (GTT). AAV.BCE.GFP was used as control.

FIGS. 33B and 33C show total body weight and accumulated body weight gain, respectively. AAV.GFP is the upper tracing and AAV.Vgll4.GFP is the lower.

FIG. 33D shows fgf21 mRNA levels in BAT following transfection with a construct driving Vgll4 with a Ucp1 cis regulatory element in accordance with aspects of the present disclosure.

FIGS. 34A and 34B show immunofluorescence images of interscapular brown adiposities (MCTI was used to label the cell borders) and Vgll4 expression, respectively, of 8-week-old C57/BL6 mice 10 days after AAV.BCE. Vgll4 or AAV.BCE.GFP was subcutaneously injected into the interscapular region.

FIGS. 34C AND 34D show real-time PCR measurements of various mRNA transcripts in brown adipose tissue. Control (AAV.BCE.GFP) is on the right and AAV.BCE. Vgll4-HF4A is on the right.

FIG. 35A shows an illustration of an experimental design demonstrating that pre-treatment with an AAV.BCE.VGLL4-HF4A mitigates body weight gain. AAV.BCE. Vgll-4HF4A was subcutaneously injected into the interscapular region of 8-weeks-old C57/BL6 mice. After 12 weeks high fat diet treatment, mice were tested for glucose (GTT) and insulin tolerance (ITT). AAV.BCE.GFP was used as control.

FIGS. 35B and 3FC show total body weight and accumulated body weight gain, respectively. FIG. 35D shows serum glucose levels following glucose challenge in a glucose tolerance test. Upper traces are control and lower traces are AAV.BCE.Vgll-4HF4A.

FIGS. 36A, 36B, 36C, 36D, AND 36E are graphs showing a ratio of brown adipose tissue weight to body weight, Vgll4 mRNA expression in BAT, Ucp1 mRNA expression in BAT, mitochondrial gene mRNA levels in BAT, and fatty acid synthesis gene mRNA expression in BAT. Student t test, *, P<0.05; **, P<0.01. Control (AAV.BCE.GFP) is on the right and AAV.BCE. Vgll4-HF4A is on the left.

FIG. 37A shows that pre-treatment with AAV.BCE. Vgll4-HF4A reduces ratio of liver weight to body weight. FIG. 37B shows liver Vgll4 mRNA expression level. FIGS. 37C and 37D are photomicrographs showing HE staining and oil red staining, respectively, of liver sections (bar=100 μm). FIG. 37E shows mRNA levels of expression of fatty acid synthesis genes in liver by quantitative real-time PCR. A, B, E, Student t test, *, P<0.05; P<0.01. Control (AAV.BCE.GFP) is on the right and AAV.BCE. Vgll-4HFA is on the right.

### DETAILED DESCRIPTION

This disclosure relates to a construct including a cis-regulatory element upstream of a coding sequence for a vestigial like 4 peptide (Vgll4). In an example, a cis-regulatory element may promote expression of a Vgll4 peptide in BAT cells. In an example, a cis-regulatory element may also promote expression of a Vgll4 peptide in liver cells. It may further specifically or enrichingly drive expression in BAT cells relative to expression driven in many or most other cells, when cells are transfected with the construct. In an example, it may further specifically or enrichingly drive expression in BAT and liver cells relative to expression driven in many or most other cells, when cells are transfected with the construct. Also disclosed is a viral vector including the construct, wherein the viral vector enables, permits, or promotes transfection of cells with the construct. In some examples, the Vgll4 peptide may include amino acid substitutions. For example, Vgll4 peptides include two TONDU (or TDU) domains, referred to herein as TDU_1 and TDU_2. Each TDU domain includes an HF dipeptide sequence. In an example, one or both HF TDU dipeptides may include amino acid substitutions, replacing HF with a dipeptide of aliphatic amino acids, such as AA.

In an example, also disclosed is causing an increase in BAT, a decrease in WAT, an increase in a ratio of BAT volume to WAT volume, or any two of the foregoing, by contacting an organism with the construct, such as by transfecting cells of an organism with the construct. In an example, also disclosed is reducing a volume of adipose tissue of the organism by contacting an organism with the construct, such as by transfecting cells of an organism with the construct. In an example, also disclosed is reducing a mass ratio BAT to body weight of an organism by contacting an organism with the construct, such as by transfecting cells of an organism with the construct. In an example, also disclosed is reducing a liver volume, liver weight, intrahepatic fat content, or any combination of two or more of the foregoing, of an organism by contacting an organism with the construct, such as by transfecting cells of an organism with the construct.

In an example, also disclosed is reducing or minimizing blood glucose levels or a rise in glucose levels in an organism by contacting an organism with the construct, such as by transfecting cells of an organism with the construct. In an example, also disclosed is increasing expression of mitochondrial genes, such as mitochondrial genes involved in mitochondrial respiration, in an organism by contacting an organism with the construct, such as by transfecting cells of an organism with the construct. In an example, also disclosed is decreasing expression of genes that promote lipogenesis, in an organism by contacting an organism with the construct, such as by transfecting cells of an organism with the construct.

In an example, a viral vector including the construct is used to transfect cells of an organism with the construct. A viral vector may be an adeno-associated viral vector or another viral vector known to be able to transfect cells. The organism may be a mammal, such as a rodent or human or any other mammal.

Vgll4 is a transcription co-factor known to interact with cellular signaling molecules and transcription factors to influence cell survival and cell function. Vgll4 is particularly known for promoting cellular death by inhibiting YAP-TEAD1 complex. Several isoforms of Vgll4 have been identified, arising from splice variants to the Vgll4 gene. These include Vgll4A, Vgll4B, Vgll4C Vgll4D, Vgll4E, and Vgll4F. Amino acid sequences of these Vgll4 proteins (referred to collectively here as Vgll4), and examples of polynucleotides encoding them encoding them, are given in Table II. Vgll4 has been linked with an anticancer effect in several types of cancer, where lower levels of Vgll4 correlate or correspond with or cause increased tumor cell survival and higher levels of Vgll4 correlate or correspond with or cause an anti-tumor effect including decreased metastatic processes and decreased tumor cell survival or proliferation. See Deng, Vgll4 is a transcriptional cofactor acting as a novel tumor suppressor via interacting with TEADs, Am J Cancer Res (2018), 8(6):932-943. In this respect, Vgll4 differs from other member of the vestigial like (Vgll) family (Vgll1, Vgl2, and Vgll3) of transcription co-factors, which are not known to have tumor-suppressive functions.

Vgll family members other than Vgll4 are not generally understood to share functional commonalities with Vgll4.

In view of the well-established role of increased Vgll4 expression in promoting cellular death processes or inhibiting cell survival, an increase in BAT volume as disclosed herein surprisingly results from Vgll4 expression influenced by a BAT-cell specific cis-regulatory element. In another example, and without being limited to any particular mechanism of action, where a Vgll4 protein includes HF to AA substitutions in both TDU domains, a reduced BAT volume, reduced intrahepatic fat accumulation, or both, may result from Vgll4 activity that does not include Vgll4 integration with a TEAD protein. In another example, and without being limited to any particular mechanism of action, where a Vgll4 protein includes HF to AA substitutions in both TDU domains, a reduced BAT volume, reduced intrahepatic fat accumulation, or both, may result from increased expression of mitochondrial genes involved in mitochondrial respiration, decreased expression of genes involved in lipogenesis, or both.

For driving expression under control of a cis-regulatory element, cis-regulatory elements of uncoupling protein 1 (Ucp1) may be placed adjacent to a coding sequence for a Vgll4. By cis-regulatory element, what is meant is a nucleotide sequence that regulates the transcription of neighboring gene or coding sequences. Conventionally, Ucp1 is considered to be expressed specifically in BAT cells. Thus, such a cis-regulatory element may drive expression mostly, or predominantly, or in some cases exclusively, in BAT cells. Surprisingly, however, as disclosed herein, such a cis-regulatory element may drive expression in liver cells in addition to expression in BAT cells. In an example, such a cis-regulatory element may drive expression only in BAT and liver cells.

A cis-regulatory element may include a promotor, an enhancer, or both. In some cases, a sequence for a cis-regulatory element may be located within fewer than 10 nucleotides from a transcription start site, fewer than 20 nucleotides from a transcription start site, fewer than 30 nucleotides from a transcription start site, fewer than 40 nucleotides from a transcription start site, fewer than 50 nucleotides from a transcription start site, fewer than 60 nucleotides from a transcription start site, fewer than 70 nucleotides from a transcription start site, fewer than 80 nucleotides from a transcription start site, fewer than 90 nucleotides from a transcription start site, fewer than 100 nucleotides from a transcription start site, fewer than 125 nucleotides from a transcription start site, fewer than 150 nucleotides from a transcription start site, fewer than 175 nucleotides from a transcription start site, fewer than 200 nucleotides from a transcription start site, fewer than 225 nucleotides from a transcription start site, fewer than 250 nucleotides from a transcription start site, fewer than 275 nucleotides from a transcription start site, fewer than 300 nucleotides from a transcription start site, fewer than 325 nucleotides from a transcription start site, fewer than 35 nucleotides from a transcription start site, fewer than 375 nucleotides from a transcription start site, fewer than 400 nucleotides from a transcription start site, fewer than 425 nucleotides from a transcription start site, fewer than 450 nucleotides from a transcription start site, fewer than 475 nucleotides from a transcription start site, fewer than 500 nucleotides from a transcription start site, or between 500 and 1,000 nucleotides from a transcription start site

A promoter is a nucleotide sequence to which RNA polymerizing enzymes bind for initiation of transcription of a downstream gene sequence. Many genes that show tissue-or cell-type specific expression including a promotor upstream of the DNA sequence that codes for the RNA that is particularly active in cells where the gene is expressed. A promoter may be more active in some cells than other, such as being active only in specific ell-or tissue-types, or highly active in certain cell-or tissue-types relative to others. Promoters include a sequence where transcription is initiated. Eukaryotic promoters may and typically do include features such as a TATA box, a transcription factor IIB recognition site, and a core promotor sequence (or an initiator). Transcription factors bind and RNA polymerase bind to a promoter for transcription initiation.

Also included in a cis-regulatory element may be one or more enhancer sequence. An enhancer is part of a cis-regulatory element that enhances transcription initiated in or by the promotor. An enhancer may serve to promote an initiation of transcription at a promoter, for example, such as through binding of additional transcription factors to the enhancer that facilitate or enhance recruitment of other factors and transcriptional machinery to the promotor. As with promotors, many genes have enhances that are involved in cell-or tissue-specific or cell-or tissue-enhanced expression.

Ucp1 is a mitochondrial protein expressed specifically in BAT cells. The Ucp1 gene includes a cis regulatory element in which enhancer and promotor elements have been identified and characterized. Such cis-regulatory elements are responsible for promoting expression of neighboring gene sequences in BAT cells and not other tissue or cell types. Sequences that may be included in a cis regulatory element in accordance with the present disclosure as based on cis regulatory elements of Ucp1 genes are shown in Table I.

TABLE I
Cis Regulatory Element Sequences

| SEQ | | |
|---|---|---|
| ID | | |
| NO | Identity | Sequence |
| 1 | Mouse | GCATGCCAATTTATAGTGCCGTCACTAACAGTACTGATACTTTAA |
| | Ucp1 | CATGCTAAGTTTAAAGTGTGTGCTATATTAATTGTAAGATTGGTG |
| | enhancer | AAGAGAGGTGTTATCAGATGGAAGCTGCACATTTCTGGATTAATG |
| | | TGGTTAAATGTATCTTCTCCTGTGATTACTGTCTTTATTTCTTCTTT |
| | | TAAAATATTGTCATTTGGACATCTATCTGTATAGCTACGCCCTGAC |
| | | ACGTCCTCCTGGAGACAGATAAGAAGTTACGACGGGAGGAGCAG |
| | | ATGGAGGCAAAGCGCTGTGATGCTTTTGTGGTTTGAGTGCACACA |
| | | TTTGTTCAGTGATTCTGTGAAATGAGTGAGCAAATGGTGACCGGG |
| | | TGCCCTGTAAATGGTGTTCTACATCTTAAGAGAAGAACACGGACA |
| | | CTAGGTAAGTGAAGCTTGCTGTCACTCCTCTACAGCGTCACAGAG |
| | | GGTCAGTCACCCTTGACCACACTGAACTAGTCGTCACCTTTCCACT |
| | | CTTCCTGCCAGAAGAGCAGAAATCAGACTCTCTGGGGATATCAGC |
| | | CTCACCCCTACTGCTCTCTCCATTATGAGGCAAACTTTCTTTCACT |
| | | TCCCAGAGGCTCTGGGGGCAGCAAGGTCAACCCTTTCCTCAGACT |
| | | CTAG |
| 2 | Mouse | TCTCGGAGGAGATCAGATCGCGCTTATTCAAGGGAACCAGCCCCT |
| | Ucp1 | GCTCTGCGCCCTGGTCCAAGGCTGTTGAAGAGTGACAAAAGGCAC |
| | promoter | CACGCTGCGGGGACGCGGGTGAAGCCCCTCTGTGTGTCCTCTGGG |
| | | CATAATCAGGAACTGGTGCCAAATCAGAGGTGATGTGGCCAGGG |
| | | CTTTGGGAGTGACGCGCGGCTGGGAGGCTTGCGCACCCAAGGCA |
| | | CGCCCCTGCCAAGTCCCACTAGCAGCTCTTTGGAGACCTGGGCCG |
| | | GCTCAGCCACTTCCCCCAGTCCCTCCTCCGGCAAGGGGCTATATA |
| | | GATCTCCCAGGTCAGGGCGCAG |
| 3 | Mouse | GCATGCCAATTTATAGTGCCGTCACTAACAGTACTGATACTTTAA |
| | Ucp1 | CATGCTAAGTTTAAAGTGTGTGCTATATTAATTGTAAGATTGGTG |
| | enhancer- | AAGAGAGGTGTTATCAGATGGAAGCTGCACATTTCTGGATTAATG |
| | promoter | TGGTTAAATGTATCTTCTCCTGTGATTACTGTCTTTATTTCTTCTTT |
| | | TAAAATATTGTCATTTGGACATCTATCTGTATAGCTACGCCCTGAC |
| | | ACGTCCTCCTGGAGACAGATAAGAAGTTACGACGGGAGGAGCAG |
| | | ATGGAGGCAAAGCGCTGTGATGCTTTTGTGGTTTGAGTGCACACA |
| | | TTTGTTCAGTGATTCTGTGAAATGAGTGAGCAAATGGTGACCGGG |
| | | TGCCCTGTAAATGGTGTTCTACATCTTAAGAGAAGAACACGGACA |
| | | CTAGGTAAGTGAAGCTTGCTGTCACTCCTCTACAGCGTCACAGAG |
| | | GGTCAGTCACCCTTGACCACACTGAACTAGTCGTCACCTTTCCACT |
| | | CTTCCTGCCAGAAGAGCAGAAATCAGACTCTCTGGGGATATCAGC |
| | | CTCACCCCTACTGCTCTCTCCATTATGAGGCAAACTTTCTTTCACT |
| | | TCCCAGAGGCTCTGGGGGCAGCAAGGTCAACCCTTTCCTCAGACT |
| | | CTAGTCTCGGAGGAGATCAGATCGCGCTTATTCAAGGGAACCAGC |
| | | CCCTGCTCTGCGCCCTGGTCCAAGGCTGTTGAAGAGTGACAAAAG |
| | | GCACCACGCTGCGGGGACGCGGGTGAAGCCCCTCTGTGTGTCCTC |
| | | TGGGCATAATCAGGAACTGGTGCCAAATCAGAGGTGATGTGGCC |
| | | AGGGCTTTGGGAGTGACGCGCGGCTGGGAGGCTTGCGCACCCAA |
| | | GGCACGCCCCTGCCAAGTCCCACTAGCAGCTCTTTGGAGACCTGG |
| | | GCCGGCTCAGCCACTTCCCCCAGTCCCTCCTCCGGCAAGGGGCTA |
| | | TATAGATCTCCCAGGTCAGGGCGCAG |
| 4 | Rat | GACGTCACAGTGGGTCAGTCACCCTTGATCACACTGCACCAGTCT |
| | adipose- | TCACCTTTCCACGCTTCCTGCCAGAGCATGAATCAGGCTCTCTGG |
| | specific | GGATACCGGCCTCACCCCTACTGAGGCAAACTTTCTCCCACTTCTC |
| | UCP1 | AGAGGCTCTGAGGGCAGCAAGGTCAGCCCTTTCTTTGGAATCTAG |
| | enhancer | AACCACTCCCTGTCTTGAGCTGACATCACAGGGCAGGCAGATGCA |
| | | GCAGGGAAGGGCCTGGGACTGGGACGTTCATCCTACAAGAAAGC |
| | | TGTGGAACTTTTCAGCAACATCTCA |
| 5 | Rat basal | GAAATCAGATCGCACTTATTCAAAGGAGCCAGGCCCTGCTCTGCG |
| | UCP1 | CCCTGGTGGAGGCTCCTCATGTGAAGAGTGACAAAAGGCACCAT |
| | promoter | GTTGTGGATACGGGGCGAAGCCCCTCCGGTGTGTCCTCCAGGCAT |
| | | CATCAGGAACTAGTGCCAAAGCAGAGGTGCTGGCCAGGGCTTTG |
| | | GGAGTGACGCGCGTCTGGGAGGCTTGTGCGCCCAGGGCACGCCC |
| | | CTGCCGATTCCCACTAGCAGGTCTTGGGGGACCTGGGCCGGCTCT |
| | | GCCCCTCCTCCAGCAATCGGGCTATAAAGCTCTTCCAAGTCAGGG |
| | | CGCAGAAGTGCCGGGCGATCCGGGCTTAAAGAGCGAGAGGAAGG |
| | | GACGCTCACCTTTGAGCTCCTCCACAAATAGCCCTGGTGGCTGCC |
| | | ACAGAAGTTCGAAGTTGAGAGTTCGG |
| 6 | Rat UCP1 | GACGTCACAGTGGGTCAGTCACCCTTGATCACACTGCACCAGTCT |
| | enhancer | TCACCTTTCCACGCTTCCTGCCAGAGCATGAATCAGGCTCTCTGG |
| | with rat | GGATACCGGCCTCACCCCTACTGAGGCAAACTTTCTCCCACTTCTC |
| | basal | AGAGGCTCTGAGGGCAGCAAGGTCAGCCCTTTCTTTGGAATCTAG |
| | UCP1 | AACCACTCCCTGTCTTGAGCTGACATCACAGGGCAGGCAGATGCA |
| | promoter | GCAGGGAAGGGCCTGGGACTGGGACGTTCATCCTACAAGAAAGC |
| | | TGTGGAACTTTTCAGCAACATCTCAGAAATCAGATCGCACTTATT |
| | | CAAAGGAGCCAGGCCCTGCTCTGCGCCCTGGTGGAGGCTCCTCAT |
| | | GTGAAGAGTGACAAAAGGCACCATGTTGTGGATACGGGGCGAAG |
| | | CCCCTCCGGTGTGTCCTCCAGGCATCATCAGGAACTAGTGCCAAA |
| | | GCAGAGGTGCTGGCCAGGGCTTTGGGAGTGACGCGCGTCTGGGA |
| | | GGCTTGTGCGCCCAGGGCACGCCCCTGCCGATTCCCACTAGCAGG |
| | | TCTTGGGGGACCTGGGCCGGCTCTGCCCCTCCTCCAGCAATCGGG |
| | | CTATAAAGCTCTTCCAAGTCAGGGCGCAGAAGTGCCGGGCGATCC |
| | | GGGCTTAAAGAGCGAGAGGAAGGGACGCTCACCTTTGAGCTCCT |
| | | CCACAAATAGCCCTGGTGGCTGCCACAGAAGTTCGAAGTTGAGA |
| | | GTTCGG |
| 7 | Human | TGATCAAGTGCATTTGTTAATGTGTTCTACATTTTCAAAAAGGAA |
| | Ucp1 | AGGAGAATTTGTTACATTCAGAACTTGCTGCCACTCCTTTGCTACG |
| | enhancer | TCATAAAGGGTCAGTTGCCCTTGCTCATACTGACCTATTCTTTACC |
| | | TCTCTGCTTCTTCTTTGTGCCAGAAGAGTAGAAATCTGACCCTTTG |
| | | GGGATACCACCCTCTCCCCTACTGCTCTCTCCAACCTGAGGCAAA |
| | | CTTTCTCCTACTTCCCAGAGCCTGTCAGAAGTGGTGAAGCCAGCC |
| | | TGCTCCTTGGAATCCAGAACTACTTTCAGAATCTTGAACTTCTGTG |
| | | ACCTCTCAGGGTCCC |
| 8 | Human | ACCGCCGCGGTGCGCCCTCCCTCCGACGTGCGGTGTGCGGGGCGC |
| | Ucp1 | AGACAACCAGCGGCCGGCCCAGGGCTTTCGGGGAGCGAAGCAGG |
| | promoter | GCTCCCGAGGCACCGAGCGAGAATGGGAATGGGAGGGACCCGGT |
| | | GCTCCCGGACACGCCCCCGGCAGGTCCCACGCCCGGGTCTTCTGA |
| | | GACCTCGCGCGGCCCAGCCCGGGAGCGGCCCAGCTATATAAGTCC |
| | | CAGCGGAAGACCGGAACGCAGAGGGTCCTGCTGGCGCGAGGGTG |
| | | GGTAGGAGGGGACGCGGGGACT |
| 9 | Human | TGATCAAGTGCATTTGTTAATGTGTTCTACATTTTCAAAAAGGAA |
| | Ucp1 | AGGAGAATTTGTTACATTCAGAACTTGCTGCCACTCCTTTGCTACG |
| | enhancer- | TCATAAAGGGTCAGTTGCCCTTGCTCATACTGACCTATTCTTTACC |
| | promoter | TCTCTGCTTCTTCTTTGTGCCAGAAGAGTAGAAATCTGACCCTTTG |
| | | GGGATACCACCCTCTCCCCTACTGCTCTCTCCAACCTGAGGCAAA |
| | | CTTTCTCCTACTTCCCAGAGCCTGTCAGAAGTGGTGAAGCCAGCC |
| | | TGCTCCTTGGAATCCAGAACTACTTTCAGAATCTTGAACTTCTGTG |
| | | ACCTCTCAGGGTCCCACCGCCGCGGTGCGCCCTCCCTCCGACGTG |
| | | CGGTGTGCGGGGCGCAGACAACCAGCGGCCGGCCCAGGGCTTTC |
| | | GGGGAGCGAAGCAGGGCTCCCGAGGCACCGAGCGAGAATGGGA |
| | | ATGGGAGGGACCCGGTGCTCCCGGACACGCCCCCGGCAGGTCCC |
| | | ACGCCCGGGTCTTCTGAGACCTCGCGCGGCCCAGCCCGGGAGCGG |
| | | CCCAGCTATATAAGTCCCAGCGGAAGACCGGAACGCAGAGGGTC |
| | | CTGCTGGCGCGAGGGTGGGTAGGAGGGGACGCGGGGACT |

Examples of Ucp1 promotors include those of SEQ ID NO: 2, SEQ ID NO: 5, and SEQ ID NO: 8 (from mouse, rat, and human Ucp1 genes, respectively). Examples of Ucp1 enhancers include those of SEQ ID NO: 1, SEQ ID NO: 4, and SEQ ID NO: 7 (from mouse, rat, and human Ucp1 genes, respectively). In an example, presence of such a Ucp1 enhancer or Ucp1 promotor, or both, or of other Ucp1 enhancer or promotor elements, or both, in the cis-regulatory element of a gene may drive transcription and expression of such gene only in BAT, or only at high levels in BAT, or only at detectable levels in BAT, or at substantially higher levels in BAT compared to other cell types. In another example, presence of such a Ucp1 enhancer or Ucp1 promotor, or both, or of other Ucp1 enhancer or promotor elements, or both, in the cis-regulatory element of a gene may also drive transcription and expression of such gene in liver cells. In some examples, a cis-regulatory element may include multiple Ucp1 enhancer elements, such as more than one of SEQ ID NO: 2, SEQ ID NO: 5, and SEQ ID NO: 8, or combination or combinations thereof.

A Ucp1 cis-regulatory element may include a sequence of SEQ ID NO: 3, SEQ ID NO: 6, or SEQ ID NO: 9. Or, it may include a Ucp1 promotor without a Ucp1 enhancer. A Ucp1 cis-regulatory element may also include combinations of a Ucp1 enhancer and a Ucp1 promotor other than the aforementioned examples, such as any one or more of enhancer SEQ ID NO: 1, SEQ ID NO: 4, and SEQ ID NO: 7, together with any one of promotor SEQ ID NO: 2, SEQ ID NO: 5, and SEQ ID NO: 8. All possible combinations and permutations of the foregoing are explicitly contemplated herein and explicitly included as examples of the present disclosure.

A cis-regulatory element including a rat Ucp1 enhancer of SEQ ID NO: 4 and a rat Ucp1 promotor of SEQ ID NO: 5 has previously been shown to drive expression of a neighboring gene in a BAT-specific manner. US Patent Application Publication No. 2016/0319303A1. As disclosed herein, a cis-regulatory element including a mouse Ucp1 enhancer of SEQ ID NO: 1, a mouse Ucp1 promotor of SEQ ID NO: 2, or both (as in SEQ ID NO: 3) may also drive expression of a neighboring gene in a BAT-specific manner. Surprisingly, as further disclosed herein, a cis-regulatory element including a Ucp1 enhancer and a Ucp1 promotor may also induce expression in liver cells. Some examples may have a Ucp1 enhancer sequence, Ucp1 promoter sequence, or Ucp1 cis-regulatory element, with 90% or more, or 95% or more, or 97.5% or more sequence homology with any of the corresponding, foregoing examples.

Amino acid sequences, and non-limiting examples of nucleotide sequences encoding such Vgll4 peptide sequences, are shown in Table II.

TABLE II
Vgl14 sequences

| SEQ | | |
|---|---|---|
| ID | Identity | |
| NO | Sequence | |
| 10 | Vgl14A | ATGCTATTTATGAAGATGGACCTGTTGAACTATCAGTACTTGGAC |
| | nucleotide | AAGATGAACAACAATATCGGCATTCTGTGCTACGAAGGCGAAGC |
| | | TGCTCTCAGGGGAGAACCCAGAATACAGACCCTGCCGGTGGCCTC |
| | | TGCCCTCAGCAGTCACCGCACCGGCCCTCCCCCAATCAGCCCCAG |
| | | CAAGAGGAAGTTCAGCATGGAGCCAGGTGACGAGGACCTAGACT |
| | | GTGACAACGACCACGTCTCCAAAATGAGTCGCATCTTCAACCCCC |
| | | ATCTGAACAAGACTGCCAATGGAGACTGCCGCAGAGACCCCCGG |
| | | GAGCGGAGCCGCAGCCCCATCGAGCGCGCTGTGGCCCCCACCAT |
| | | GAGCCTGCACGGCAGCCACCTGTACACCTCCCTCCCCAGCCTTGG |
| | | CCTGGAGCAGCCCCTCGCACTGACCAAGAACAGCCTGGACGCCA |
| | | GCAGGCCAGCCGGCCTCTCGCCCACACTGACCCCGGGGGAGCGG |
| | | CAGCAGAACCGGCCCTCCGTGATCACCTGTGCCTCGGCTGGCGCC |
| | | CGCAACTGCAACCTCTCGCACTGCCCCATCGCGCACAGCGGCTGT |
| | | GCCGCGCCCGGGCCTGCCAGCTACCGGAGGCCACCGAGCGCTGC |
| | | CACCACCTGTGACCCCGTGGTGGAGGAGCATTTCCGCAGGAGCCT |
| | | GGGCAAGAATTACAAGGAGCCCGAGCCGGCACCCAACTCCGTGT |
| | | CCATCACGGGCTCCGTGGACGACCACTTTGCCAAAGCTCTGGGTG |
| | | ACACGTGGCTCCAGATCAAAGCGGCCAAGGACGGAGCATCCAGC |
| | | AGCCCTGAGTCCGCCTCTCGCAGGGGCCAGCCCGCCAGCCCCTCT |
| | | GCCCACATGGTCAGCCACAGTCACTCCCCCTCTGTGGTCTCC |
| 11 | Vgl14A | MLFMKMDLLNYQYLDKMNNNIGILCYEGEAALRGEPRIQTLPVASA |
| | amino acid | LSSHRTGPPPISPSKRKFSMEPGDEDLDCDNDHVSKMSRIFNPHLNKT |
| | | ANGDCRRDPRERSRSPIERAVAPTMSLHGSHLYTSLPSLGLEQPLALT |
| | | KNSLDASRPAGLSPTLTPGERQQNRPSVITCASAGARNCNLSHCPIAH |
| | | SGCAAPGPASYRRPPSAATTCDPVVEEHFRRSLGKNYKEPEPAPNSV |
| | | SITGSVDDHFAKALGDTWLQIKAAKDGASSSPESASRRGQPASPSAH |
| | | MVSHSHSPSVVS |
| 12 | Vgl14B | ATGGAGACGCCATTGGATGTTTTGTCCAGGGCAGCATCTCTGGTG |
| | nucleotide | CATGCTGATGACGAAAAACGCGAAGCTGCTCTCAGGGGAGAACC |
| | | CAGAATACAGACCCTGCCGGTGGCCTCTGCCCTCAGCAGTCACCG |
| | | CACCGGCCCTCCCCCAATCAGCCCCAGCAAGAGGAAGTTCAGCAT |
| | | GGAGCCAGGTGACGAGGACCTAGACTGTGACAACGACCACGTCT |
| | | CCAAAATGAGTCGCATCTTCAACCCCCATCTGAACAAGACTGCCA |
| | | ATGGAGACTGCCGCAGAGACCCCCGGGAGCGGAGCCGCAGCCCC |
| | | ATCGAGCGCGCTGTGGCCCCCACCATGAGCCTGCACGGCAGCCAC |
| | | CTGTACACCTCCCTCCCCAGCCTTGGCCTGGAGCAGCCCCTCGCA |
| | | CTGACCAAGAACAGCCTGGACGCCAGCAGGCCAGCCGGCCTCTC |
| | | GCCCACACTGACCCCGGGGGAGCGGCAGCAGAACCGGCCCTCCG |
| | | TGATCACCTGTGCCTCGGCTGGCGCCCGCAACTGCAACCTCTCGC |
| | | ACTGCCCCATCGCGCACAGCGGCTGTGCCGCGCCCGGGCCTGCCA |
| | | GCTACCGGAGGCCACCGAGCGCTGCCACCACCTGTGACCCCGTGG |
| | | TGGAGGAGCATTTCCGCAGGAGCCTGGGCAAGAATTACAAGGAG |
| | | CCCGAGCCGGCACCCAACTCCGTGTCCATCACGGGCTCCGTGGAC |
| | | GACCACTTTGCCAAAGCTCTGGGTGACACGTGGCTCCAGATCAAA |
| | | GCGGCCAAGGACGGAGCATCCAGCAGCCCTGAGTCCGCCTCTCGC |
| | | AGGGGCCAGCCCGCCAGCCCCTCTGCCCACATGGTCAGCCACAGT |
| | | CACTCCCCCTCTGTGGTCTCC |
| 13 | Vgl14B | METPLDVLSRAASLVHADDEKREAALRGEPRIQTLPVASALSSHRTG |
| | peptide | PPPISPSKRKFSMEPGDEDLDCDNDHVSKMSRIFNPHLNKTANGDCR |
| | | RDPRERSRSPIERAVAPTMSLHGSHLYTSLPSLGLEQPLALTKNSLDA |
| | | SRPAGLSPTLTPGERQQNRPSVITCASAGARNCNLSHCPIAHSGCAAP |
| | | GPASYRRPPSAATTCDPVVEEHFRRSLGKNYKEPEPAPNSVSITGSVD |
| | | DHFAKALGDTWLQIKAAKDGASSSPESASRRGQPASPSAHMVSHSH |
| | | SPSVVS |
| 14 | Vgl14C | ATGATTAAAGTGAGGAACAAGACTGCCAATGGAGACTGCCGCAG |
| | nucleotide | AGACCCCCGGGAGCGGAGCCGCAGCCCCATCGAGCGCGCTGTGG |
| | | CCCCCACCATGAGCCTGCACGGCAGCCACCTGTACACCTCCCTCC |
| | | CCAGCCTTGGCCTGGAGCAGCCCCTCGCACTGACCAAGAACAGCC |
| | | TGGACGCCAGCAGGCCAGCCGGCCTCTCGCCCACACTGACCCCGG |
| | | GGGAGCGGCAGCAGAACCGGCCCTCCGTGATCACCTGTGCCTCGG |
| | | CTGGCGCCCGCAACTGCAACCTCTCGCACTGCCCCATCGCGCACA |
| | | GCGGCTGTGCCGCGCCCGGGCCTGCCAGCTACCGGAGGCCACCG |
| | | AGCGCTGCCACCACCTGTGACCCCGTGGTGGAGGAGCATTTCCGC |
| | | AGGAGCCTGGGCAAGAATTACAAGGAGCCCGAGCCGGCACCCAA |
| | | CTCCGTGTCCATCACGGGCTCCGTGGACGACCACTTTGCCAAAGC |
| | | TCTGGGTGACACGTGGCTCCAGATCAAAGCGGCCAAGGACGGAG |
| | | CATCCAGCAGCCCTGAGTCCGCCTCTCGCAGGGGCCAGCCCGCCA |
| | | GCCCCTCTGCCCACATGGTCAGCCACAGTCACTCCCCCTCTGTGGT |
| | | CTCC |
| 15 | Vgl14C | MIKVRNKTANGDCRRDPRERSRSPIERAVAPTMSLHGSHLYTSLPSL |
| | amino acid | GLEQPLALTKNSLDASRPAGLSPTLTPGERQQNRPSVITCASAGARNC |
| | | NLSHCPIAHSGCAAPGPASYRRPPSAATTCDPVVEEHFRRSLGKNYK |
| | | EPEPAPNSVSITGSVDDHFAKALGDTWLQIKAAKDGASSSPESASRR |
| | | GQPASPSAHMVSHSHSPSVVS |
| 16 | Vgl14D | ATGAACAAGACTGCCAATGGAGACTGCCGCAGAGACCCCCGGGA |
| | nucleotide | GCGGAGCCGCAGCCCCATCGAGCGCGCTGTGGCCCCCACCATGA |
| | | GCCTGCACGGCAGCCACCTGTACACCTCCCTCCCCAGCCTTGGCC |
| | | TGGAGCAGCCCCTCGCACTGACCAAGAACAGCCTGGACGCCAGC |
| | | AGGCCAGCCGGCCTCTCGCCCACACTGACCCCGGGGGAGCGGCA |
| | | GCAGAACCGGCCCTCCGTGATCACCTGTGCCTCGGCTGGCGCCCG |
| | | CAACTGCAACCTCTCGCACTGCCCCATCGCGCACAGCGGCTGTGC |
| | | CGCGCCCGGGCCTGCCAGCTACCGGAGGCCACCGAGCGCTGCCA |
| | | CCACCTGTGACCCCGTGGTGGAGGAGCATTTCCGCAGGAGCCTGG |
| | | GCAAGAATTACAAGGAGCCCGAGCCGGCACCCAACTCCGTGTCC |
| | | ATCACGGGCTCCGTGGACGACCACTTTGCCAAAGCTCTGGGTGAC |
| | | ACGTGGCTCCAGATCAAAGCGGCCAAGGACGGAGCATCCAGCAG |
| | | CCCTGAGTCCGCCTCTCGCAGGGGCCAGCCCGCCAGCCCCTCTGC |
| | | CCACATGGTCAGCCACAGTCACTCCCCCTCTGTGGTCTCC |
| 17 | Vgl14D | MNKTANGDCRRDPRERSRSPIERAVAPTMSLHGSHLYTSLPSLGLEQ |
| | amino acid | PLALTKNSLDASRPAGLSPTLTPGERQQNRPSVITCASAGARNCNLSH |
| | | CPIAHSGCAAPGPASYRRPPSAATTCDPVVEEHFRRSLGKNYKEPEPA |
| | | PNSVSITGSVDDHFAKALGDTWLQIKAAKDGASSSPESASRRGQPAS |
| | | PSAHMVSHSHSPSVVS |
| 18 | Vgl14E | ATGACTGAGAATACGCATTTTGACAAAATCCCTGAGTCCTGTGCA |
| | nucleotide | CTCAAAAGTTGGAGACATCCAGGTCTGCACCATGGCGAAGCTGCT |
| | | CTCAGGGGAGAACCCAGAATACAGACCCTGCCGGTGGCCTCTGCC |
| | | CTCAGCAGTCACCGCACCGGCCCTCCCCCAATCAGCCCCAGCAAG |
| | | AGGAAGTTCAGCATGGAGCCAGGTGACGAGGACCTAGACTGTGA |
| | | CAACGACCACGTCTCCAAAATGAGTCGCATCTTCAACCCCCATCT |
| | | GAACAAGACTGCCAATGGAGACTGCCGCAGAGACCCCCGGGAGC |
| | | GGAGCCGCAGCCCCATCGAGCGCGCTGTGGCCCCCACCATGAGCC |
| | | TGCACGGCAGCCACCTGTACACCTCCCTCCCCAGCCTTGGCCTGG |
| | | AGCAGCCCCTCGCACTGACCAAGAACAGCCTGGACGCCAGCAGG |
| | | CCAGCCGGCCTCTCGCCCACACTGACCCCGGGGGAGCGGCAGCA |
| | | GAACCGGCCCTCCGTGATCACCTGTGCCTCGGCTGGCGCCCGCAA |
| | | CTGCAACCTCTCGCACTGCCCCATCGCGCACAGCGGCTGTGCCGC |
| | | GCCCGGGCCTGCCAGCTACCGGAGGCCACCGAGCGCTGCCACCA |
| | | CCTGTGACCCCGTGGTGGAGGAGCATTTCCGCAGGAGCCTGGGCA |
| | | AGAATTACAAGGAGCCCGAGCCGGCACCCAACTCCGTGTCCATCA |
| | | CGGGCTCCGTGGACGACCACTTTGCCAAAGCTCTGGGTGACACGT |
| | | GGCTCCAGATCAAAGCGGCCAAGGACGGAGCATCCAGCAGCCCT |
| | | GAGTCCGCCTCTCGCAGGGGCCAGCCCGCCAGCCCCTCTGCCCAC |
| | | ATGGTCAGCCACAGTCACTCCCCCTCTGTGGTCTCC |
| 19 | Vgl14E | MTENTHFDKIPESCALKSWRHPGLHHGEAALRGEPRIQTLPVASALS |
| | amino acid | SHRTGPPPISPSKRKFSMEPGDEDLDCDNDHVSKMSRIFNPHLNKTA |
| | | NGDCRRDPRERSRSPIERAVAPTMSLHGSHLYTSLPSLGLEQPLALTK |
| | | NSLDASRPAGLSPTLTPGERQQNRPSVITCASAGARNCNLSHCPIAHS |
| | | GCAAPGPASYRRPPSAATTCDPVVEEHFRRSLGKNYKEPEPAPNSVSI |
| | | TGSVDDHFAKALGDTWLQIKAAKDGASSSPESASRRGQPASPSAHM |
| | | VSHSHSPSVVS |
| 20 | Vgl14F | ATGGAGCCAGGTGACGAGGACCTAGACTGTGACAACGACCACGT |
| | nucleotide | CTCCAAAATGAGTCGCATCTTCAACCCCCATCTGAACAAGACTGC |
| | | CAATGGAGACTGCCGCAGAGACCCCCGGGAGCGGAGCCGCAGCC |
| | | CCATCGAGCGCGCTGTGGCCCCCACCATGAGCCTGCACGGCAGCC |
| | | ACCTGTACACCTCCCTCCCCAGCCTTGGCCTGGAGCAGCCCCTCG |
| | | CACTGACCAAGAACAGCCTGGACGCCAGCAGGCCAGCCGGCCTC |
| | | TCGCCCACACTGACCCCGGGGGAGCGGCAGCAGAACCGGCCCTC |
| | | CGTGATCACCTGTGCCTCGGCTGGCGCCCGCAACTGCAACCTCTC |
| | | GCACTGCCCCATCGCGCACAGCGGCTGTGCCGCGCCCGGGCCTGC |
| | | CAGCTACCGGAGGCCACCGAGCGCTGCCACCACCTGTGACCCCGT |
| | | GGTGGAGGAGCATTTCCGCAGGAGCCTGGGCAAGAATTACAAGG |
| | | AGCCCGAGCCGGCACCCAACTCCGTGTCCATCACGGGCTCCGTGG |
| | | ACGACCACTTTGCCAAAGCTCTGGGTGACACGTGGCTCCAGATCA |
| | | AAGCGGCCAAGGACGGAGCATCCAGCAGCCCTGAGTCCGCCTCT |
| | | CGCAGGGGCCAGCCCGCCAGCCCCTCTGCCCACATGGTCAGCCAC |
| | | AGTCACTCCCCCTCTGTGGTCTCC |
| 21 | Vgl14F | MEPGDEDLDCDNDHVSKMSRIFNPHLNKTANGDCRRDPRERSRSPIE |
| | amino acid | RAVAPTMSLHGSHLYTSLPSLGLEQPLALTKNSLDASRPAGLSPTLTP |
| | | GERQQNRPSVITCASAGARNCNLSHCPIAHSGCAAPGPASYRRPPSA |
| | | ATTCDPVVEEHFRRSLGKNYKEPEPAPNSVSITGSVDDHFAKALGDT |
| | | WLQIKAAKDGASSSPESASRRGQPASPSAHMVSHSHSPSVVS |

At least six isoforms (A-F) of Vgll4 have been identified, referred to herein as Vgll4A, Vgll4B, Vgll4C, Vgll4D, Vgll4E, and Vgll4F, having amino acid sequences SEQ ID NO 11, SEQ ID NO 13, SEQ ID NO 15, SEQ ID NO 17, SEQ ID NO 19, and SEQ ID NO 21, respectively. These six isoforms are collectively included in the term Vgll4 as used herein. Also included herein is any nucleotide sequence that encodes any of the foregoing Vgll4 isoforms, including, without limitation, SEQ ID NO 10, SEQ ID NO: 12, SEQ ID NO 14, SEQ ID NO 16, SEQ ID NO 18, and SEQ ID NO: 20, including one or more codon substitution to any of the foregoing nucleotide sequences that nevertheless still encodes a Vgl4 (e.g., A-F), owing to codon degeneracy. A construct as disclosed herein may include a nucleotide sequence encoding a Vgll4 peptide as disclosed herein with any cis-regulatory element as disclosed herein, including without limitation one or more Ucp1 enhancer and a Ucp1 promotor, including any variation thereof described above.

A Vgll4 protein may be of human Vgll4 (SEQ ID NO 11, SEQ ID NO 13, SEQ ID NO 15, SEQ ID NO 17, SEQ ID NO 19, or SEQ ID NO 21), or mouse or rat Vgll4, or a Vgll4 sequence having at least 90%, at least 95%, or at least 97.5% homology with any of the foregoing examples in Table II. In an example, A Vgll4 peptide may include one or more amino acid substitution (relative to the examples disclosed in Table II). In an example, a Vgll4 peptide may include 1, 2, 3, 4, 5, 6, 7, 8, 9, 10, 11, 12, 13, 14, 15, 16, 17, 18, 19, or 20 amino acid substitutions (relative to the examples disclosed in Table II). In an example, a Vgll4 peptide may have from 1 to 3 amino acid substitutions, or 2 amino acid substitutions, or 1 amino acid substitutions (relative to the examples disclosed in Table II). In an example, any of the foregoing amino acid substitutions may be outside of a TDU_1 and TDU_2 domain of the Vgll4 peptide.

In another example, a Vgll4 peptide may include two amino acid substitutions an a TDU domain, or two amino acid substitutions in each of two TDU domains. A TDU_1 domain has the amino acid sequence SEQ ID NO: 41. A TDU_2 domain has the amino acid sequence SEQ ID NO: 42. Each of SEQ ID NO 11, SEQ ID NO 13, SEQ ID NO 15, SEQ ID NO 17, SEQ ID NO 19, and SEQ ID NO 21 includes a TDU_1 domain with amino acid sequence SEQ ID NO: 41 and a TDU_2 domain with amino acid sequence SEQ ID NO: 42.

In some examples, a TDU_1 domain may have an HF dipeptide amino acid sequence substituted with an AA dipeptide amino acid sequence, to yield the TDU_1 amino acid sequence SEQ ID NO: 43. In some examples, a TDU_2 domain may have an HF dipeptide amino acid sequence substituted with an AA dipeptide amino acid sequence, to yield the TDU_2 amino acid sequence SEQ ID NO: 44. A Vgll4 peptide may include a TDU_1 domain having an amino acid sequence of SEQ ID NO: 43 instead of an amino acid sequence of SEQ ID NO: 41. A Vgll4 peptide may include a TDU_2 domain having an amino acid sequence of SEQ ID NO: 44 instead of an amino acid sequence of SEQ ID NO: 42. A Vgll peptide may include a TDU_1 domain having an amino acid sequence of SEQ ID NO: 43 instead of an amino acid sequence of SEQ ID NO: 41 and include a TDU_2 domain having an amino acid sequence of SEQ ID NO: 44 instead of an amino acid sequence of SEQ ID NO: 42. A Vgll4 peptide (e.g., Vgll4A, Vgll4B, Vgll4C, Vgll4D, Vgll4E, or Vgll4F) having TDU domains with amino acid sequences SEQ ID NO: 43 and SEQ ID NO: 44 instead of SEQ ID NO: 41 and SEQ ID NO: 42 is referred to herein as a Vgll4-HF4A peptide.

At least six isoforms (A-F) of Vgll4-HF4A are disclosed herein, referred to herein as Vgll4A-HF4A, Vgll4B-HF4A, Vgll4C-HF4A, Vgll4D-HF4A, Vgll4E-HF4A, and Vgll4F-HF4A. Amino acid sequences, and non-limiting examples of nucleotide sequences encoding such Vgll4-HF4A peptide sequences, are shown in Table III.

TABLE III
Vgl14-HF4A sequences

| SEQ | | |
|---|---|---|
| ID | | |
| NO | Identity | Sequence |
| 22 | Vgl14A- | ATGCTATTTATGAAGATGGACCTGTTGAACTATCAGTACTTGGAC |
| | HF4A | AAGATGAACAACAATATCGGCATTCTGTGCTACGAAGGCGAAGC |
| | nucleotide | TGCTCTCAGGGGAGAACCCAGAATACAGACCCTGCCGGTGGCCTC |
| | | TGCCCTCAGCAGTCACCGCACCGGCCCTCCCCCAATCAGCCCCAG |
| | | CAAGAGGAAGTTCAGCATGGAGCCAGGTGACGAGGACCTAGACT |
| | | GTGACAACGACCACGTCTCCAAAATGAGTCGCATCTTCAACCCCC |
| | | ATCTGAACAAGACTGCCAATGGAGACTGCCGCAGAGACCCCCGG |
| | | GAGCGGAGCCGCAGCCCCATCGAGCGCGCTGTGGCCCCCACCAT |
| | | GAGCCTGCACGGCAGCCACCTGTACACCTCCCTCCCCAGCCTTGG |
| | | CCTGGAGCAGCCCCTCGCACTGACCAAGAACAGCCTGGACGCCA |
| | | GCAGGCCAGCCGGCCTCTCGCCCACACTGACCCCGGGGGAGCGG |
| | | CAGCAGAACCGGCCCTCCGTGATCACCTGTGCCTCGGCTGGCGCC |
| | | CGCAACTGCAACCTCTCGCACTGCCCCATCGCGCACAGCGGCTGT |
| | | GCCGCGCCCGGGCCTGCCAGCTACCGGAGGCCACCGAGCGCTGC |
| | | CACCACCTGTGACCCCGTGGTGGAGGAGGCAGCCCGCAGGAGCC |
| | | TGGGCAAGAATTACAAGGAGCCCGAGCCGGCACCCAACTCCGTG |
| | | TCCATCACGGGCTCCGTGGACGACGCAGCTGCCAAAGCTCTGGGT |
| | | GACACGTGGCTCCAGATCAAAGCGGCCAAGGACGGAGCATCCAG |
| | | CAGCCCTGAGTCCGCCTCTCGCAGGGGCCAGCCCGCCAGCCCCTC |
| | | TGCCCACATGGTCAGCCACAGTCACTCCCCCTCTGTGGTCTCC |
| 23 | Vgl14A- | MLFMKMDLLNYQYLDKMNNNIGILCYEGEAALRGEPRIQTLPVASA |
| | HF4A | LSSHRTGPPPISPSKRKFSMEPGDEDLDCDNDHVSKMSRIFNPHLNKT |
| | amino acid | ANGDCRRDPRERSRSPIERAVAPTMSLHGSHLYTSLPSLGLEQPLALT |
| | | KNSLDASRPAGLSPTLTPGERQQNRPSVITCASAGARNCNLSHCPIAH |
| | | SGCAAPGPASYRRPPSAATTCDPVVEEAARRSLGKNYKEPEPAPNSV |
| | | SITGSVDDAAAKALGDTWLQIKAAKDGASSSPESASRRGQPASPSAH |
| | | MVSHSHSPSVVS |
| 24 | Vgl14B- | ATGGAGACGCCATTGGATGTTTTGTCCAGGGCAGCATCTCTGGTG |
| | HF4A | CATGCTGATGACGAAAAACGCGAAGCTGCTCTCAGGGGAGAACC |
| | nucleotide | CAGAATACAGACCCTGCCGGTGGCCTCTGCCCTCAGCAGTCACCG |
| | | CACCGGCCCTCCCCCAATCAGCCCCAGCAAGAGGAAGTTCAGCAT |
| | | GGAGCCAGGTGACGAGGACCTAGACTGTGACAACGACCACGTCT |
| | | CCAAAATGAGTCGCATCTTCAACCCCCATCTGAACAAGACTGCCA |
| | | ATGGAGACTGCCGCAGAGACCCCCGGGAGCGGAGCCGCAGCCCC |
| | | ATCGAGCGCGCTGTGGCCCCCACCATGAGCCTGCACGGCAGCCAC |
| | | CTGTACACCTCCCTCCCCAGCCTTGGCCTGGAGCAGCCCCTCGCA |
| | | CTGACCAAGAACAGCCTGGACGCCAGCAGGCCAGCCGGCCTCTC |
| | | GCCCACACTGACCCCGGGGGAGCGGCAGCAGAACCGGCCCTCCG |
| | | TGATCACCTGTGCCTCGGCTGGCGCCCGCAACTGCAACCTCTCGC |
| | | ACTGCCCCATCGCGCACAGCGGCTGTGCCGCGCCCGGGCCTGCCA |
| | | GCTACCGGAGGCCACCGAGCGCTGCCACCACCTGTGACCCCGTGG |
| | | TGGAGGAGGCAGCCCGCAGGAGCCTGGGCAAGAATTACAAGGAG |
| | | CCCGAGCCGGCACCCAACTCCGTGTCCATCACGGGCTCCGTGGAC |
| | | GACGCAGCTGCCAAAGCTCTGGGTGACACGTGGCTCCAGATCAA |
| | | AGCGGCCAAGGACGGAGCATCCAGCAGCCCTGAGTCCGCCTCTC |
| | | GCAGGGGCCAGCCCGCCAGCCCCTCTGCCCACATGGTCAGCCACA |
| | | GTCACTCCCCCTCTGTGGTCTCC |
| 25 | Vgl14B- | METPLDVLSRAASLVHADDEKREAALRGEPRIQTLPVASALSSHRTG |
| | HF4A | PPPISPSKRKFSMEPGDEDLDCDNDHVSKMSRIFNPHLNKTANGDCR |
| | peptide | RDPRERSRSPIERAVAPTMSLHGSHLYTSLPSLGLEQPLALTKNSLDA |
| | | SRPAGLSPTLTPGERQQNRPSVITCASAGARNCNLSHCPIAHSGCAAP |
| | | GPASYRRPPSAATTCDPVVEEAARRSLGKNYKEPEPAPNSVSITGSVD |
| | | DAAAKALGDTWLQIKAAKDGASSSPESASRRGQPASPSAHMVSHSH |
| | | SPSVVS |
| 26 | Vgl14C- | ATGATTAAAGTGAGGAACAAGACTGCCAATGGAGACTGCCGCAG |
| | HF4A | AGACCCCCGGGAGCGGAGCCGCAGCCCCATCGAGCGCGCTGTGG |
| | nucleotide | CCCCCACCATGAGCCTGCACGGCAGCCACCTGTACACCTCCCTCC |
| | | CCAGCCTTGGCCTGGAGCAGCCCCTCGCACTGACCAAGAACAGCC |
| | | TGGACGCCAGCAGGCCAGCCGGCCTCTCGCCCACACTGACCCCGG |
| | | GGGAGCGGCAGCAGAACCGGCCCTCCGTGATCACCTGTGCCTCGG |
| | | CTGGCGCCCGCAACTGCAACCTCTCGCACTGCCCCATCGCGCACA |
| | | GCGGCTGTGCCGCGCCCGGGCCTGCCAGCTACCGGAGGCCACCG |
| | | AGCGCTGCCACCACCTGTGACCCCGTGGTGGAGGAGGCAGCCCG |
| | | CAGGAGCCTGGGCAAGAATTACAAGGAGCCCGAGCCGGCACCCA |
| | | ACTCCGTGTCCATCACGGGCTCCGTGGACGACGCAGCTGCCAAAG |
| | | CTCTGGGTGACACGTGGCTCCAGATCAAAGCGGCCAAGGACGGA |
| | | GCATCCAGCAGCCCTGAGTCCGCCTCTCGCAGGGGCCAGCCCGCC |
| | | AGCCCCTCTGCCCACATGGTCAGCCACAGTCACTCCCCCTCTGTG |
| | | GTCTCC |
| 27 | Vgl14C- | MIKVRNKTANGDCRRDPRERSRSPIERAVAPTMSLHGSHLYTSLPSL |
| | HF4A | GLEQPLALTKNSLDASRPAGLSPTLTPGERQQNRPSVITCASAGARNC |
| | amino acid | NLSHCPIAHSGCAAPGPASYRRPPSAATTCDPVVEEAARRSLGKNYK |
| | | EPEPAPNSVSITGSVDDAAAKALGDTWLQIKAAKDGASSSPESASRR |
| | | GQPASPSAHMVSHSHSPSVVS |
| 28 | Vgl14D- | ATGAACAAGACTGCCAATGGAGACTGCCGCAGAGACCCCCGGGA |
| | HF4A | GCGGAGCCGCAGCCCCATCGAGCGCGCTGTGGCCCCCACCATGA |
| | nucleotide | GCCTGCACGGCAGCCACCTGTACACCTCCCTCCCCAGCCTTGGCC |
| | | TGGAGCAGCCCCTCGCACTGACCAAGAACAGCCTGGACGCCAGC |
| | | AGGCCAGCCGGCCTCTCGCCCACACTGACCCCGGGGGAGCGGCA |
| | | GCAGAACCGGCCCTCCGTGATCACCTGTGCCTCGGCTGGCGCCCG |
| | | CAACTGCAACCTCTCGCACTGCCCCATCGCGCACAGCGGCTGTGC |
| | | CGCGCCCGGGCCTGCCAGCTACCGGAGGCCACCGAGCGCTGCCA |
| | | CCACCTGTGACCCCGTGGTGGAGGAGGCAGCCCGCAGGAGCCTG |
| | | GGCAAGAATTACAAGGAGCCCGAGCCGGCACCCAACTCCGTGTC |
| | | CATCACGGGCTCCGTGGACGACGCAGCTGCCAAAGCTCTGGGTGA |
| | | CACGTGGCTCCAGATCAAAGCGGCCAAGGACGGAGCATCCAGCA |
| | | GCCCTGAGTCCGCCTCTCGCAGGGGCCAGCCCGCCAGCCCCTCTG |
| | | CCCACATGGTCAGCCACAGTCACTCCCCCTCTGTGGTCTCC |
| 29 | Vgl14D- | MNKTANGDCRRDPRERSRSPIERAVAPTMSLHGSHLYTSLPSLGLEQ |
| | HF4A | PLALTKNSLDASRPAGLSPTLTPGERQQNRPSVITCASAGARNCNLSH |
| | amino acid | CPIAHSGCAAPGPASYRRPPSAATTCDPVVEEAARRSLGKNYKEPEP |
| | | APNSVSITGSVDDAAAKALGDTWLQIKAAKDGASSSPESASRRGQPA |
| | | SPSAHMVSHSHSPSVVS |
| 30 | Vgl14E- | ATGACTGAGAATACGCATTTTGACAAAATCCCTGAGTCCTGTGCA |
| | HF4A | CTCAAAAGTTGGAGACATCCAGGTCTGCACCATGGCGAAGCTGCT |
| | nucleotide | CTCAGGGGAGAACCCAGAATACAGACCCTGCCGGTGGCCTCTGCC |
| | | CTCAGCAGTCACCGCACCGGCCCTCCCCCAATCAGCCCCAGCAAG |
| | | AGGAAGTTCAGCATGGAGCCAGGTGACGAGGACCTAGACTGTGA |
| | | CAACGACCACGTCTCCAAAATGAGTCGCATCTTCAACCCCCATCT |
| | | GAACAAGACTGCCAATGGAGACTGCCGCAGAGACCCCCGGGAGC |
| | | GGAGCCGCAGCCCCATCGAGCGCGCTGTGGCCCCCACCATGAGCC |
| | | TGCACGGCAGCCACCTGTACACCTCCCTCCCCAGCCTTGGCCTGG |
| | | AGCAGCCCCTCGCACTGACCAAGAACAGCCTGGACGCCAGCAGG |
| | | CCAGCCGGCCTCTCGCCCACACTGACCCCGGGGGAGCGGCAGCA |
| | | GAACCGGCCCTCCGTGATCACCTGTGCCTCGGCTGGCGCCCGCAA |
| | | CTGCAACCTCTCGCACTGCCCCATCGCGCACAGCGGCTGTGCCGC |
| | | GCCCGGGCCTGCCAGCTACCGGAGGCCACCGAGCGCTGCCACCA |
| | | CCTGTGACCCCGTGGTGGAGGAGGCAGCCCGCAGGAGCCTGGGC |
| | | AAGAATTACAAGGAGCCCGAGCCGGCACCCAACTCCGTGTCCATC |
| | | ACGGGCTCCGTGGACGACGCAGCTGCCAAAGCTCTGGGTGACAC |
| | | GTGGCTCCAGATCAAAGCGGCCAAGGACGGAGCATCCAGCAGCC |
| | | CTGAGTCCGCCTCTCGCAGGGGCCAGCCCGCCAGCCCCTCTGCCC |
| | | ACATGGTCAGCCACAGTCACTCCCCCTCTGTGGTCTCC |
| 31 | Vgl14E- | MTENTHFDKIPESCALKSWRHPGLHHGEAALRGEPRIQTLPVASALS |
| | HF4A | SHRTGPPPISPSKRKFSMEPGDEDLDCDNDHVSKMSRIFNPHLNKTA |
| | amino acid | NGDCRRDPRERSRSPIERAVAPTMSLHGSHLYTSLPSLGLEQPLALTK |
| | | NSLDASRPAGLSPTLTPGERQQNRPSVITCASAGARNCNLSHCPIAHS |
| | | GCAAPGPASYRRPPSAATTCDPVVEEAARRSLGKNYKEPEPAPNSVS |
| | | ITGSVDDAAAKALGDTWLQIKAAKDGASSSPESASRRGQPASPSAH |
| | | MVSHSHSPSVVS |
| 32 | Vgl14F- | ATGGAGCCAGGTGACGAGGACCTAGACTGTGACAACGACCACGT |
| | HF4A | CTCCAAAATGAGTCGCATCTTCAACCCCCATCTGAACAAGACTGC |
| | nucleotide | CAATGGAGACTGCCGCAGAGACCCCCGGGAGCGGAGCCGCAGCC |
| | | CCATCGAGCGCGCTGTGGCCCCCACCATGAGCCTGCACGGCAGCC |
| | | ACCTGTACACCTCCCTCCCCAGCCTTGGCCTGGAGCAGCCCCTCG |
| | | CACTGACCAAGAACAGCCTGGACGCCAGCAGGCCAGCCGGCCTC |
| | | TCGCCCACACTGACCCCGGGGGAGCGGCAGCAGAACCGGCCCTC |
| | | CGTGATCACCTGTGCCTCGGCTGGCGCCCGCAACTGCAACCTCTC |
| | | GCACTGCCCCATCGCGCACAGCGGCTGTGCCGCGCCCGGGCCTGC |
| | | CAGCTACCGGAGGCCACCGAGCGCTGCCACCACCTGTGACCCCGT |
| | | GGTGGAGGAGGCAGCCCGCAGGAGCCTGGGCAAGAATTACAAGG |
| | | AGCCCGAGCCGGCACCCAACTCCGTGTCCATCACGGGCTCCGTGG |
| | | ACGACGCAGCTGCCAAAGCTCTGGGTGACACGTGGCTCCAGATCA |
| | | AAGCGGCCAAGGACGGAGCATCCAGCAGCCCTGAGTCCGCCTCT |
| | | CGCAGGGGCCAGCCCGCCAGCCCCTCTGCCCACATGGTCAGCCAC |
| | | AGTCACTCCCCCTCTGTGGTCTCC |
| 33 | Vgl14F- | MEPGDEDLDCDNDHVSKMSRIFNPHLNKTANGDCRRDPRERSRSPIE |
| | HF4A | RAVAPTMSLHGSHLYTSLPSLGLEQPLALTKNSLDASRPAGLSPTLTP |
| | amino acid | GERQQNRPSVITCASAGARNCNLSHCPIAHSGCAAPGPASYRRPPSA |
| | | ATTCDPVVEEAARRSLGKNYKEPEPAPNSVSITGSVDDAAAKALGDT |
| | | WLQIKAAKDGASSSPESASRRGQPASPSAHMVSHSHSPSVVS |

As disclosed herein, a Vgll4-HF4A peptide may have an amino acid sequence of s SEQ ID NO 23, SEQ ID NO 25, SEQ ID NO 27, SEQ ID NO 29, SEQ ID NO 31, or SEQ ID NO 33. These six isoforms are collectively included in the term Vgll4-HF4A as used herein. Also included herein is any nucleotide sequence that encodes any of the foregoing Vgll4 isoforms, including, without limitation, SEQ ID NO 22, SEQ ID NO: 24, SEQ ID NO 26, SEQ ID NO 28, SEQ ID NO 30, and SEQ ID NO: 32, including one or more codon substitution to any of the foregoing nucleotide sequences that nevertheless still encodes a Vgl4-HF4A (e.g., A-F), owing to codon degeneracy. A construct as disclosed herein may include a nucleotide sequence encoding a Vgll4-HF4A peptide as disclosed herein with any cis-regulatory element as disclosed herein, including without limitation one or more Ucp1 enhancer and a Ucp1 promotor, including any variation thereof described above.

A Vgll4-HF4A protein may be a human Vgll4, or mouse or rat Vgll4, bearing an HF to AA substitution in its TDU domains, or a Vgll4-HF4A sequence having at least 90%, at least 95%, or at least 97.5% homology with any of the foregoing examples in Table III. In an example, a Vgll4-HF4A peptide may include one or more amino acid substitution (relative to the examples disclosed in Table III) outside a TDU_1 and TDU_2 domain. In an example, a Vgll4-HF4A peptide may include 1, 2, 3, 4, 5, 6, 7, 8, 9, 10, 11, 12, 13, 14, 15, 16 17, 18, 19, or 20 amino acid substitutions (relative to sequences disclosed in Table III) outside a TDU_1 and TDU_2 domain. In an example, a Vgll4-HF4A peptide may have from 1 to 3 amino acid substitutions, or 2 amino acid substitutions, or 1 amino acid substitution (relative to sequences disclosed in Table III) outside a TDU_1 and TDU_2 domain.

In some examples, an intron may be included between a cis-regulatory element and a gene encoding Vgll4. In some examples, an intron may enhance or promote transcription or promote stability of an RNA transcript. Other examples do not include an intron. Various intronic sequences are known by skilled artisans to be able to be included in recombinant constructs for promoting gene expression, any of which could be included in a construct as disclosed herein. In an example, an intron of SEQ ID NO: 34 (a chimeric intron of human b-globin and immunoglobulin heavy chain genes) may be included, or a sequence having at least 90%, at least 95%, or at least 97.5% sequence homology therewith.

A summary of aspects of a construct including a cis regulatory element and a Vgll4-encoding nucleotide sequence, and a cis regulatory element and a Vgll4-HF4A-encoding nucleotide sequence, as disclosed herein are shown in Tables IV and V, respectively.

TABLE IV
Vgl14 constructs

| cis-Regulatory Elements | | Optional intron | Vgl14 |
|---|---|---|---|
| Enhancer sequences | Promoter sequences | SEQ ID NO: 34 | Amino acid sequences: |
| SEQ ID NO: 1 | SEQ ID NO: 2 | GTAAGTATCAAGG | SEQ ID NO: 11 |
| SEQ ID NO: 4 | SEQ ID NO: 5 | TTACAAGACAGGT | SEQ ID NO: 13 |
| SEQ ID NO: 7 | SEQ ID NO: 8 | TTAAGGAGACCAA | SEQ ID NO: 15 |

| | | |
|---|---|---|
| Enhancer-promoter sequences: | TAGAAACTGGGCT | SEQ ID NO: 17 |
| SEQ ID NO: 3 | TGTCGAGACAGAG | SEQ ID NO: 19 |
| SEQ ID NO: 6 | AAGACTCTTGCGTT | SEQ ID NO: 21 |
| SEQ ID NO: 9 | TCTGATAGGCACCT | Nucleotide sequences: |
| | ATTGGTCTTACTGA | SEQ ID NO: 10 |
| | CATCCACTTTGCCT | SEQ ID NO: 12 |
| | TTCTCTCCACAG | SEQ ID NO: 14 |
| | | SEQ ID NO: 16 |
| | | SEQ ID NO: 18 |
| | | SEQ ID NO: 20 |

TABLE V
Vgl14-HF4A constructs

| cis-Regulatory Elements | | Optional intron | Vgl14-HF4A |
|---|---|---|---|
| Enhancer sequences | Promoter sequences | SEQ ID NO: 34 | Amino acid sequences: |
| SEQ ID NO: 1 | SEQ ID NO: 2 | GTAAGTATCAAGG | SEQ ID NO: 23 |
| SEQ ID NO: 4 | SEQ ID NO: 5 | TTACAAGACAGGT | SEQ ID NO: 25 |
| SEQ ID NO: 7 | SEQ ID NO: 8 | TTAAGGAGACCAA | SEQ ID NO: 27 |

| | | |
|---|---|---|
| Enhancer-promoter sequences: | TAGAAACTGGGCT | SEQ ID NO: 29 |
| SEQ ID NO: 3 | TGTCGAGACAGAG | SEQ ID NO: 31 |
| SEQ ID NO: 6 | AAGACTCTTGCGTT | SEQ ID NO: 33 |
| SEQ ID NO: 9 | TCTGATAGGCACCT | Nucleotide sequences: |
| | ATTGGTCTTACTGA | SEQ ID NO: 22 |
| | CATCCACTTTGCCT | SEQ ID NO: 24 |
| | TTCTCTCCACAG | SEQ ID NO: 26 |
| | | SEQ ID NO: 28 |
| | | SEQ ID NO: 30 |
| | | SEQ ID NO: 32 |

A construct as disclosed herein may include a cis regulatory element and a nucleotide sequence encoding a Vgll4 or Vgll4-HF4A peptide. A cis regulatory element may include, for example, any one or more of enhancer sequence SEQ ID NO: 1, SEQ ID NO: 4, and SEQ ID NO: 7, and any one of promoter sequence SEQ ID NO: 2, SEQ ID NO: 5, and SEQ ID NO: 8. All permutations of the foregoing are expressly contemplated and included in the present disclosure. In an example, a cis-regulatory element may include 2, 3, or 4 enhancer sequences each independently selected from SEQ ID NO: 1, SEQ ID NO: 4, and SEQ ID NO: 7. Examples of cis regulatory elements include SEQ ID NO: 3, SEQ ID NO: 6, and SEQ ID NO: 9.

A Vgll4 peptide encoded by a Vgll4 peptide-encoding nucleotide sequence of a construct may include, for example, any of SEQ ID NO: 11, SEQ ID NO: 13, SEQ ID NO: 15, SEQ ID NO: 17, SEQ ID NO: 19, and SEQ ID NO: 21, or any variation thereof as further explained above. Examples include SEQ ID NO: 10, SEQ ID NO: 12, SEQ ID NO: 14, SEQ ID NO: 16, SEQ ID NO: 18, and SEQ ID NO: 20. Vgll4-HF4A peptide encoded by a Vgll4-HF4A peptide-encoding nucleotide sequence of a construct may include, for example, any of SEQ ID NO: 23, SEQ ID NO: 25, SEQ ID NO: 27, SEQ ID NO: 29, SEQ ID NO: 31, and SEQ ID NO: 31, or any variation thereof as further explained above. Examples include SEQ ID NO: 22, SEQ ID NO: 24, SEQ ID NO: 26, SEQ ID NO: 28, SEQ ID NO: 30, and SEQ ID NO: 32. A construct may include any cis regulatory element as disclosed herein and any Vgll4- or Vgll-4HF4A-encoding nucleotide sequence as disclosed herein. Optionally, a construct may also include an intron between a cis regulatory element and a Vgll4- or Vgll4-HF4A-encoding nucleotide sequence. A non-limiting example of an optional intron is SEQ ID NO: 34. In other examples, nucleotides other than an intron or having an intronic nucleotide sequence other than SEQ ID NO: 34 may be included in a construct between a cis regulatory element and a Vgll4- or Vgll4-HF4A-encoding nucleotide sequence. An example of a Vgll4A construct is SEQ ID NO: 35 and an example of a Vgll4-HF4A construct is SEQ ID NO: 36.

A cell may be transfected with a construct as disclosed above by various methods, such as chemical transfection, electroporation, impalefaction, gene gun transfection, or viral vector mediated gene transfer, or any other method known to skilled persons in the relevant field. In an example, a Vgll4 gene with associated Ucp1 cis-regulatory element is packaged in a viral vector for cellular transfection. Viral vector in this case refers to a viral-like particle that contains or includes a payload gene construct or cassette capable of attaching to a cell and delivering the payload into the cell. In some examples, a viral vector may be of a type wherein a payload, once introduced into a transfected cell, integrates into the cell's genomic DNA, though such genomic integration is not an essential feature of a viral vector as disclosed herein. Viral vector may also refer to a gene sequence including a gene construct or cassette structured for inclusion in a viral-like particle. Examples of viral vectors include retroviruses, lentiviruses, adenoviruses, and adeno-associated viruses (AAV). Several serotypes of AAV vectors are useful for cellular transfection, including any of serotypes 1, 2, 3, 4, 5, 6, 7, 8, 9, 10, and 11, or subtypes thereof. Sequences for such AAV serotypes are known and may be found in publicly accessible databases, as are methods of packaging a construct of interest in viral vector particles for cellular transfection and promotion of construct expression in transfected cells.

An AAV vector includes sequences bounding a payload construct referred to as inverted terminal repeats (ITRs). ITR sequences are involved in transcription of AAV genome, encapsulation of payload in a vector particle, genome multiplication for particle generation, and integration into host genome. A cassette, construct, transgene, payload, etc., placed between ITRs of an AAV vector may promote production of an AAV vector and/or expression of transfected gene within cells. In an example, a Ucp1 cis-regulatory element neighboring a nucleotide sequence encoding a Vgll4 or Vgll4-HFA peptide may be placed between ITRs and used for generation of an AAV particle, wherein said particle may be contacted with cells of an organism to transfect them with such construct. An example includes an AAV9 serotype AAV containing such construct, though other serotypes may also be used.

In some cases a reporter gene may be used or included in a construct to verify expression of a construct gene included in a vector or for testing tissue-or cell-type specific expression of a gene under control of a given cis-regulatory element. Numerous reporter genes are known and have been widely used in the relevant field. A non-limiting list of examples includes a green fluorescent protein (for example, having an amino acid sequence of SEQ ID NO: 37, encoded for by a nucleotide sequence of SEQ ID NO: 38 or any other nucleotide sequence as may encode an amino acid sequence of SEQ ID NO: 37 according to principles codon degeneracy), a yellow fluorescent protein, a red fluorescent protein, a blue fluorescent protein, a luciferase protein, a beta-galactosidase protein, a glutathione S-transferase protein, a chloramphenicol acetyltransferase protein, and any combination of two or more of the foregoing. Other reporters may also be included. In other examples, no reporter is included. By detecting expression of a reporter protein, the ability of a given cis-regulatory element, or viral vector, to promote transfection and/or expression in various cell ad tissue types can be evaluated. A reporter protein sequence may occur immediately before the N-terminal or immediately after the C-terminal amino acid of a Vgll4 or Vgll4-HF4A peptide as disclosed herein, or may be separated by and one or more amino acids from the N-or C-terminal amino acid of a Vgll4 or Vgll4-HF4A peptide. A construct may include any nucleotide sequence for encoding any reporter protein. A non-limiting example of a Vgll4 construct including a sequence encoding a GFP-encoding reported protein is SEQ ID NO: 39. In another non-limiting example, SEQ ID NO: 39 may be modified to replace SEQ ID NO: 41 therein and SEQ ID NO: 42 therein with SEQ ID NO: 43 and SEQ ID NO: 44, respectively, to encode a Vgll4-HF4A and a GFP reporter protein.

A viral vector or viral-like particle, such as an AAV vector, can be injected into an organism, such as subcutaneously, intramuscularly, intravenously, intraperitoneally, or by other methods for introduction of the vector into the organism for contact with cells thereof. A vector may contact various different cell and tissue types and transfect them. However, inclusion of a cell-or tissue-specific cis-regulatory element (enhancer, promotor, or both) may restrict expression of the transfected gene to a given cell or tissue type or types, wherein the construct is not transcribed or is otherwise dormant or at most barely or minimally expressed in other cell types. As explained above, a cis-regulatory element may include elements that are known or believed to drive expression in adipocytes, or fat cells, specifically, including in specific subtypes of fat cells, such as predominantly in BAT cells. However, it is not necessary that expression be limited absolutely to a given cell type, including only in BAT cells, even under control of a Ucp1 cis-regulatory element. For example, although Ucp1 expression is believed to be restricted to mature BAT cells, it is possible that other cell types may from time to time express a construct whose expression is influenced by a neighboring cis-regulatory element such as a Ucp1 enhancer, promoter, or both. Surprisingly, in some circumstances, as disclosed herein, a Ucp1 cis-regulatory element may include expression in liver cells in addition to BAT cells.

In an example, contacting the cells of an organism with a nucleotide sequence including a Ucp1 cis-regulatory element and a coding sequence for a Vgll4 protein or variant may increase BAT volume, lower WAT volume, increase a ratio of BAT volume to WAT volume, or any combination of the foregoing, even if Vgll4 expression under control of the cis-regulatory element is not strictly limited to BAT cells containing the transgene. In another example, a construct may

In another example, transfecting cells of an organism with a nucleotide sequence including a Ucp1 cis-regulatory element and a coding sequence for a Vgll4 protein or variant may reduce a volume of adipose tissue of the organism. In another example, transfecting cells of an organism with a nucleotide sequence including a Ucp1 cis-regulatory element and a coding sequence for a Vgll4 protein or variant may reduce a mass ratio BAT to body weight of the organism.

In another example, disclosed herein is a method for prevention or treatment of obesity, by transfecting cells of an organism with any of the foregoing constructs disclosed herein including a cis-regulatory element and nucleotide sequence encoding a Vgll4 peptide or a Vgll4-HF4A peptide.

In another example, transfecting cells of an organism with a nucleotide sequence including a Ucp1 cis-regulatory element and a coding sequence for a Vgll4 protein or variant may reduce a liver volume, liver weight, intrahepatic fat content, or any combination of two or more of the foregoing, of the organism. An intrahepatic fat content of at least 5% of liver weight is referred to as hepatic steatosis. Obesity, or risk of developing obesity, such as genetic or life-style factors (e.g., high-calorie or high-fat diet, low exercise or caloric burn rate, sedentary lifestyle, etc.), are risk factors for developing elevated hepatic steatosis. Obesity may be defined as having a body mass index (BMI) of 30 or higher. An example of a risk factor for developing obesity may be having a BMI of from 25 to 29, which is considered being overweight. Prolonged hepatic steatosis is is a risk factor for disorders including liver metabolic dysfunction, inflammation, and advanced forms of nonalcoholic fatty liver disease. Disclosed herein is a method for prevention or treatment of hepatic steatosis, by transfecting cells of an organism with any of the foregoing constructs disclosed herein including a cis-regulatory element and nucleotide sequence encoding a Vgll4 peptide or a Vgll4-HF4A peptide.

In another example, transfecting cells of an organism with a nucleotide sequence including a Ucp1 cis-regulatory element and a coding sequence for a Vgll4 protein or variant may reduce or minimize blood glucose levels or a rise in glucose levels or duration of such rise in an organism. Obesity is a risk factor for diabetes, which includes pathological dysregulation of glucose levels, specifically pathological elevations in serum glucose levels or pathologically elevated duration of elevated serum glucose levels such as following calorie intake such as a meal. In an example, a rise in serum glucose may be measured following administration of a glucose challenge (i.e., consuming a glucose solution). Normally, a rise in serum glucose follows such a challenge, which rise then returns to baseline or near baseline. In individuals with diabetes, however, glucose may rise pathologically higher and/or for a pathologically longer duration than in individuals without diabetes. For individuals with diabetes or at risk for developing diabetes (e.g., family history, genetic or other biomarker-evinced predisposition, obesity, etc.), a treatment for preventing pathological rise in serum glucose levels, or a pathological extension of a rise in serum glucose levels, following a meal or a glucose challenge is advantageous. An example in accordance with the present disclosure includes reducing or minimizing blood glucose levels or a rise in glucose levels in an organism by contacting an organism with the construct, such as by transfecting cells of an organism with the construct. An example in accordance with the present disclosure includes preventing development of a pathologically high rise in blood glucose levels or a pathologically high duration of a rise in blood glucose levels in an organism with diabetes or at risk for developing diabetes by contacting an organism with the construct, such as by transfecting cells of the organism. The organism may be an obese person, or a person at risk of developing obesity, or a person diagnosed with diabetes, or a person at risk of developing diabetes. Accordingly, an example disclosed herein includes a method for prevention or treatment of diabetes, by transfecting cells of an organism with any of the foregoing constructs disclosed herein including a cis-regulatory element and nucleotide sequence encoding a Vgll4 peptide or a Vgll4-HF4A peptide.

In an example, also disclosed is increasing expression of mitochondrial genes, such as mitochondrial genes involved in mitochondrial respiration, in an organism by contacting an organism with the construct, such as by transfecting cells of an organism with the construct. In an example, also disclosed is decreasing expression of genes that promote lipogenesis, in an organism by contacting an organism with the construct, such as by transfecting cells of an organism with the construct. Increasing mitochondrial genes that promote mitochondrial respiration, or decreasing expression of genes involved in lipogenesis, such as in BAT or liver cells of an organism transfected with a construct as disclosed herein (e.g., including a Ucp1 cis regulatory element and a nucleotide sequence encoding a Vgll4-or Vgll4-HFA-encoding nucleotide, as disclosed herein), may include advantageously promote BAT levels, decrease lipogenesis in adipose cells, decrease hepatic steatosis, or any combination of the foregoing.

As disclosed herein, in an example, contacting cells of an organism with a nucleotide sequence including a Ucp1 cis-regulatory element neighboring a Vgll4 coding sequence surprisingly increases BAT volume, i.e. the volume occupied by BAT cells. In another example, contacting cells of an organism with a nucleotide sequence including a Ucp1 cis-regulatory element neighboring a Vgll4 coding sequence surprisingly decreases WAT volume. In another example, contacting cells of an organism with a nucleotide sequence including a Ucp1 cis-regulatory element neighboring a Vgll4 coding sequence surprisingly increases a ration of BAT volume to WAT volume. An increase in BAT by driving Vgll4 expression under control of a Ucp1 cis-regulatory element, known to increase expression of a neighboring gene in BAT cells, is particularly unexpected given that increased Vgll4 expression is known to promote apoptosis or otherwise have anti-tumor cell effects, unlike other members of the Vgll family. By comparison, Vgll3 levels are increased in WAT cells in obese mice, suggesting that Vgll3 may promote WAT cells, whereas over-expression of Vgl13 inhibits adipogenesis overall. U.S. Pat. No. 8,852,939.

As further disclosed herein, in an example, contacting cells of an organism with a nucleotide sequence including a Ucp1 cis-regulatory element neighboring a Vgll4 coding sequence or a Vgll4-HF4A sequence in some cases may promote expression of Vgll4 or Vgll4-HF4A respectively, in liver.

In another example, contacting cells of an organism with a nucleotide sequence including a Ucp1 cis-regulatory element neighboring a Vgll4-HF4A coding sequence may surprisingly decreases adipose tissue volume. In an example, a volume of BAT is decreased. Without limiting the present disclosure to any particular mechanism of action, decreased volume of BAT following transfection with such a construct may be related to stimulation of expression of mitochondrial genes involved in mitochondrial respiration, inhibition of expression of genes involved in lipogenesis, an inhibition of lipogenesis, or any combination of two or more of the foregoing, in BAT, caused by the transfection. In another example, hepatic steatosis is decreased. Without limiting the present disclosure to any particular mechanism of action, decreased hepatic steatosis following transfection with such a construct may be related to stimulation of expression of mitochondrial genes involved in mitochondrial respiration, inhibition of expression of genes involved in lipogenesis, an inhibition of lipogenesis, or any combination of two or more of the foregoing, in liver, caused by the transfection.

### EXAMPLES

The following examples are intended to illustrate particular embodiments of the present disclosure, but are by no means intended to limit the scope thereof.

### Example 1: A Ucp1 Cis-Regulatory Element Drives Expression in BAT When Transfection Occurs During Early Development

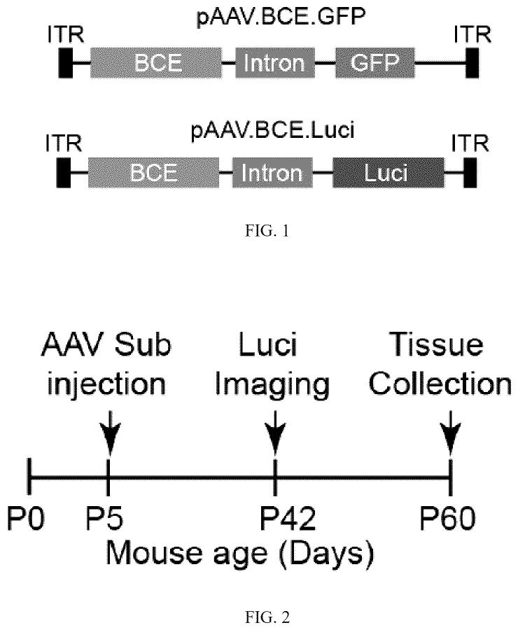

FIG. 1 shows examples of polynucleotides in accordance with certain aspects of the present disclosure. The example includes a cis-regulatory element (BCE) including a mouse Ucp1 enhancer and mouse Ucp1 promoter upstream of a coding sequence of a reporter protein (either a green fluorescent protein (GFP) of firefly luciferase (Luci)). In these examples, a chimeric intron of human b-globin and immunoglobulin heavy chain genes is included between the BCE and the reporter sequence. Inclusion of an intron may increase expression of the payload gene sequence, here the reporter constructs. Also included at the 5′ and 3′ ends, flanking the BCE and reporter, are inverted terminal repeat (ITR) sequences. Adeno-associated viral vectors were synthesized incorporating constructs as illustrated for determining an ability of BCE to drive expression of a downstream coding sequence. In an example, adeno-associated viral vectors of serotype 9 (AAV9) were constructed carrying constructs as illustrated in FIG. 1. Constructs include a BCE having a sequence of SEQ ID NO: 3 and an intron of SEQ ID NO: 34.

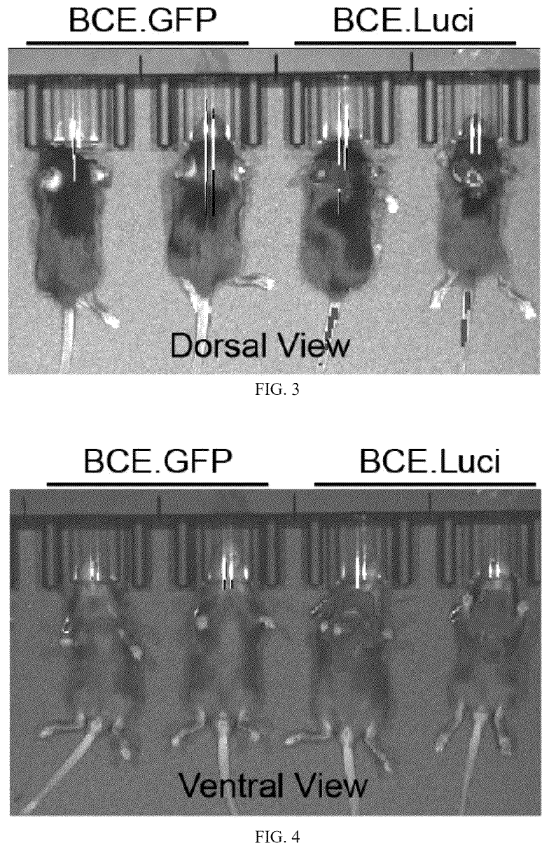

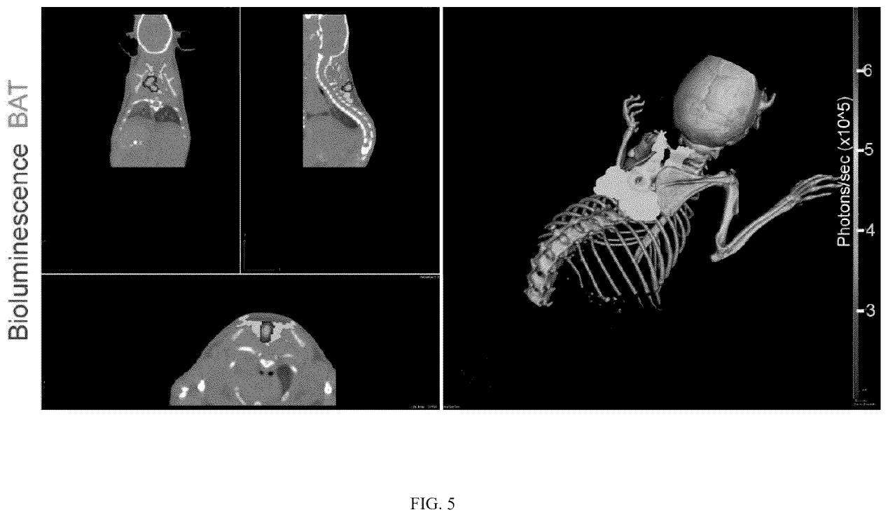

FIG. 2 illustrates time course of treatment of mice with a viral vectors as illustrated in FIG. 1 for determining expression patterns of reporter proteins driven by BCE. At 5 days of age (P5), neonatal mice received dorsal sc AAV injection (of an AAV9 carrying one of the constructs shown in FIG. 1), at a dose of 1×10¹⁰ genome copies per gram of body weight, in phosphate-buffered saline. At 42 days after birth (P42), bioluminescence was assessed to determine expression of marker proteins. Expression was determined by an in vivo imaging system (IVIS™, Perkin Elmer) as shown in FIGS. 3 (dorsal view) and 4 (ventral view), as well as by micro CT scanning to show topographical expression patterns, as shown in FIG. 5. AAV9.BCE.luci transduced subjects were first imaged for bioluminescence, and then scanned by micro CT, with AAV9.BCE. GFP serving as a control. In FIG. 5, bioluminescence signal origins were matched with tissues mapped by micro CT (pink=brown adipose tissue, blue or red=bioluminescence signal positive tissues). Expression was specifically elevated in BAT, demonstrating the BAT-specific expression driven by the BCE cis-regulatory element following transfection during the neonatal period.

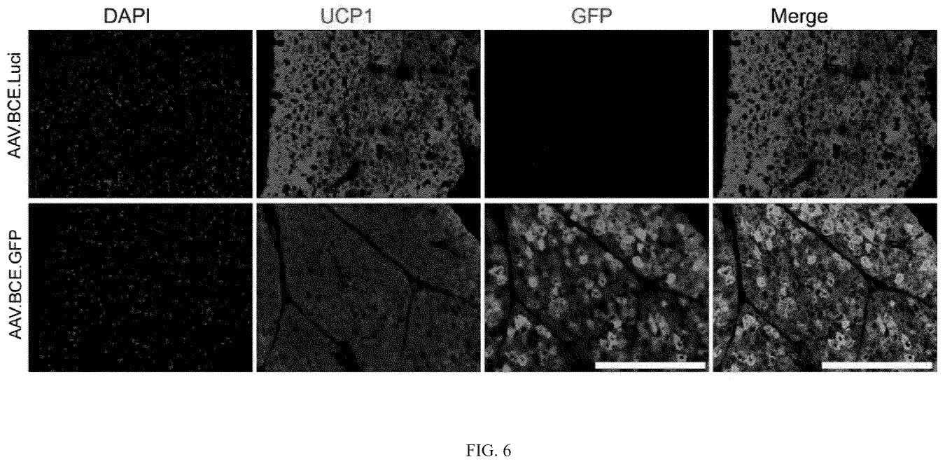

As shown in FIGS. 6 and 7, interscapular adipose tissues (i.e., in the region where BAT is located) were collected at postnatal day 60 and used for immunofluorescence staining. FIG. 6 shows immunofluorescence staining images of interscapular BAT (using UCP1 imaging to identify BAT cells), and FIG. 7 shows immunofluorescence staining images of both BAT and white adipose tissue (using perilipin staining to mark adipose tissue, both BAT and WAT.). Nuclear stain DAPI is also shown in FIG. 6 and GFP expression was used to stain cells with BCE-driven reporter expression in animals treated with AAV.BCE.GFP (with AAV.BCE.Luci treatment serving as control). Bar=200 μm. BCE drove expression of reporter protein in adipocytes, and in BAT in particular.

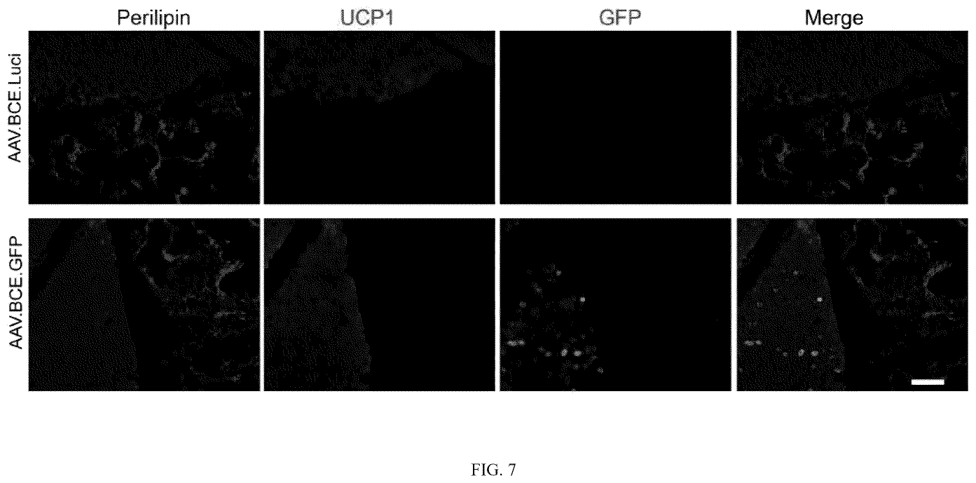

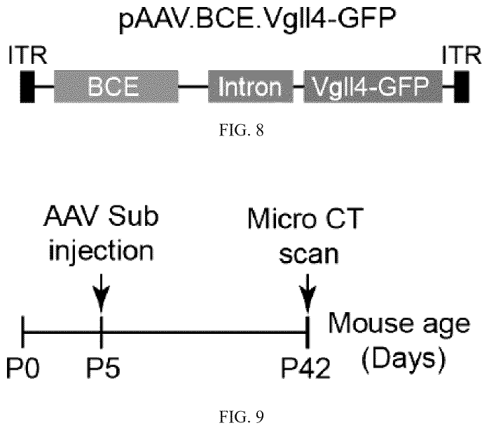

FIG. 8 shows a schematic view of a pAAV.BCE. Vgll4-GFP construct administered via an AAV9 carrier. The construct resembles that shown in FIG. 1 except that Vgll4-GFP is the coding sequence whose expression is driven by BCE rather than merely GFP or luciferase. The sequence of the BCA-intron-Vgll4-GFP transcript is SEQ ID NO:39, and is a polynucleotide including a cis-regulatory element and a nucleotide sequence encoding a vestigial like 4 protein, wherein the cis-regulatory element includes an uncoupling protein 1 enhancer and an uncoupling protein 1 promoter. An intron is present between the cis-regulatory element and nucleotide sequence encoding a vestigial like 4 protein. The construct was packaged in an adeno-associated viral vector (AAV9) for transfection of cells of an organism with the construct.

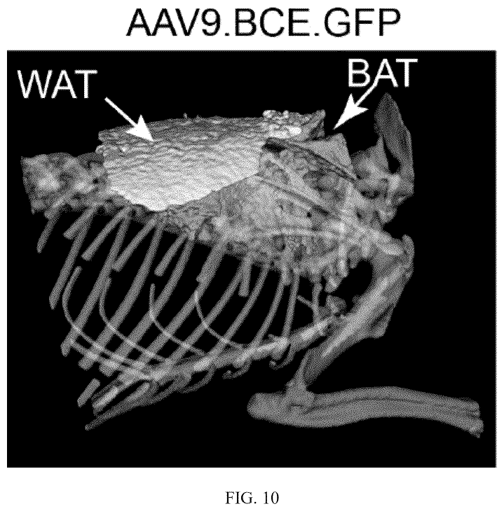

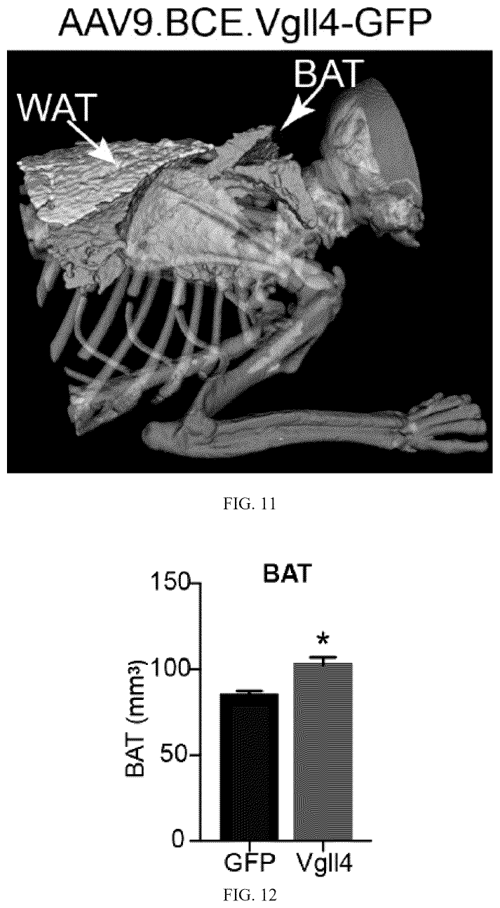

FIG. 9 illustrates time course of treatment of mice with a viral vectors as illustrated in FIG. 8 for determining effects on adipose tissue volume. On postnatal day 5 mice were injected dorsally sc with AAV (AAV9.BCE. Vgll4-GFP, or AAV9.BCE.GFP, carrying the construct shown in FIG. 1, as a control), at a dose of 1×10¹⁰ genome copies per gram of body weight, in phosphate-buffered saline. On postnatal day 42, a micro CT scan was taken to measure intrascapular adipose tissue volume, using standard commercially available micro CT scan software (analyze 12™). Intrascapular adipose tissue was differentiable from neighboring tissue, and BAT was differentiable from WAT, due to differences in Hounsfield units associated with differing tissue types according to standard micro CT scan techniques. An example scan is shown in FIGS. 10 (control) and 11 (for a subject injected with AAV9.BCE. Vgll4-GFP). BAT and WAT are as indicated. Increased volume of BAT and decreased volume of WAT can be seen following BCE-driven expression of Vgll4.

BAT volume was measured as graphically represented in FIG. 12. In subjects with BCE-driven Vgll4 expression, BAT volume was an average of 102.2±2.8 mm³, compared to 84.2±1.9 mm³ in controls (*=p<0.05). WAT volume was measured as graphically represented in FIG. 13. In subjects with BCE-driven Vgll4 expression, WAT volume was an average of 27.1±0.6 mm³, compared to 40.0±2.8 mm³ in controls (**=p<0.01). Ratio of BAT/WAT volume was measured as graphically represented in FIG. 14. In subjects with BCE-driven Vgll4 expression, BAT/WAT volume ration was an average of 3.8±0.6 mm³, compared to 40.0±2.8 mm³ in controls (**=p<0.01). Thus, transfection of cells of an organism with a construct including a cis-regulatory element driving expression of Vgll4, wherein the cis-regulatory element includes an uncoupling protein 1 enhancer and an uncoupling protein 1 promoter, increased BAT volume, decreased WAT volume, and increased a ration of BAT volume to WAT volume.

### Example 2: A Ucp1 Cis-Regulatory Element Drives Expression in BAT and Liver When Transfection Occurs During Adulthood

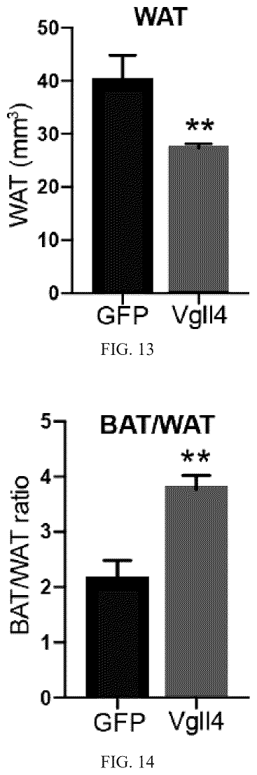

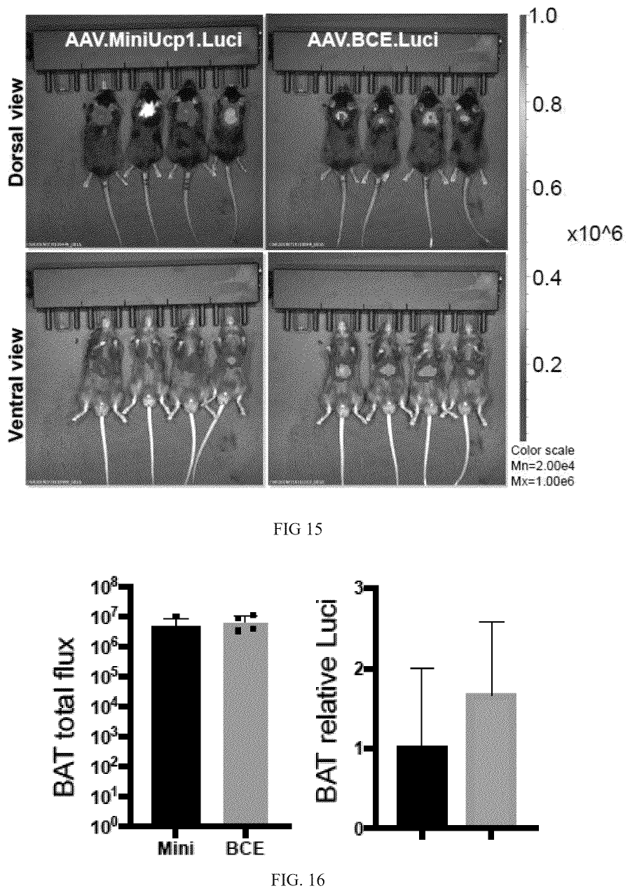

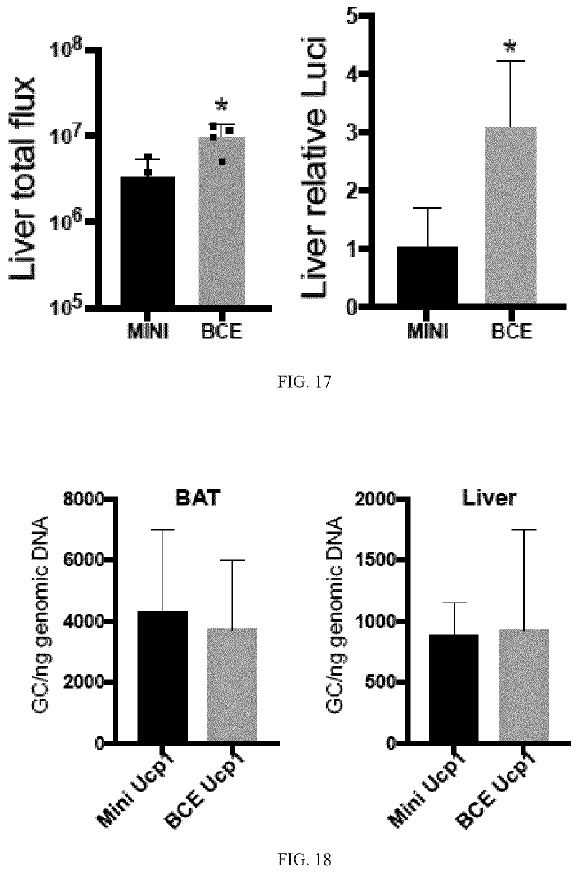

An AAV vector (AAV9) containing a construct with a BCE cis regulatory element (SEQ ID NO: 3) driving expression of luciferase (AAV.BCE.Luci) was administered to to 6 weeks old mice. In other mice, the cis regulatory element (MiniUcp1) included a Ucp1 enhancer (SEQ ID NO: 4) but not a Ucp1 promoter (AAV.MiniUcp1.Luci). Subjects were tested for luciferase signals one week later. Results are illustrated in FIGS. 15-18. Both BAT (FIG. 16) and liver (FIG. 17) had luciferase signals, with BCE cis regulatory element driving higher liver expression that MiniUcp1. AAV.BCE.Luci drives expression in liver when transfection occurs later in development such as in adulthood. FIG. 18 shows that BAT and liver included viral vector genome copies, as assessed by real-time PCR.

AAV has very low chance of integrating into the host cell genome, existing primarily as episomes in host cells. If the host cells proliferate rapidly, daughter cells may easily lose AAV copy number. In cell cycle quiescent cells, in contrast, AAV coexist until cells die. During development, such as in neonates, hepatocytes are rapidly proliferating, whereas BAT are mostly cell cycle quiescent cells. Without being limited to any particular theory or mechanism of action, during the growth of AAV.BCE.Luci transduced pups, hepatocytes but not the brown adipocytes may have shed AAVs, which could explain why luciferase signals can only be detected in the BAT but not in the liver following transfection early in development (as in Example 1). In the AAV.BCE. Luci transduced adult mice of the present Example (Example 2), BAT and hepatocytes were not actively proliferating, and luciferase signals were detected in both tissues.

### Example 3. The Hippo-YAP Pathway

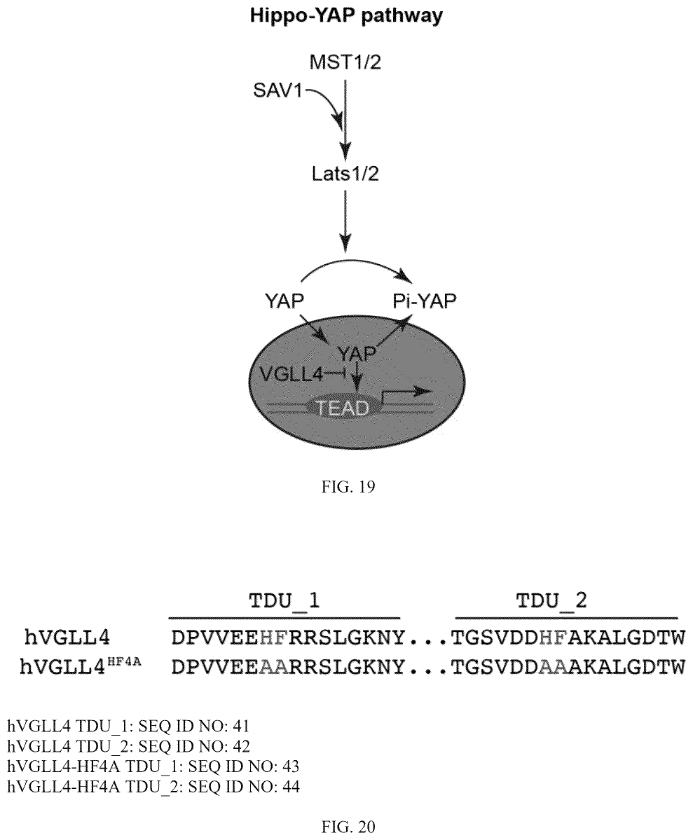

The Hippo-YAP signaling pathway is well known for controlling organ growth. In mammals, the Hippo kinase cascade includes MST1/2, LATS1/2, and the scaffold protein Salvador (Sav). Activation of these kinases results in phosphorylation and inactivation of YAP and WWTR1 (more commonly known as TAZ), orthologous transcriptional coactivators that are terminal effectors of this pathway. YAP/TAZ interact with TEAD family transcription factors to regulate downstream target genes expression. Vestigial like 4 (VGLL4) is another co-transcriptional factor that serves as a suppressor of a YAP-TEAD complex. Mechanistically, VGLL4 directly binds to TEAD through its two TONDU (TDU) domains, and the binding of VGLL4 or YAP to TEAD is mutually exclusive (FIG. 19).

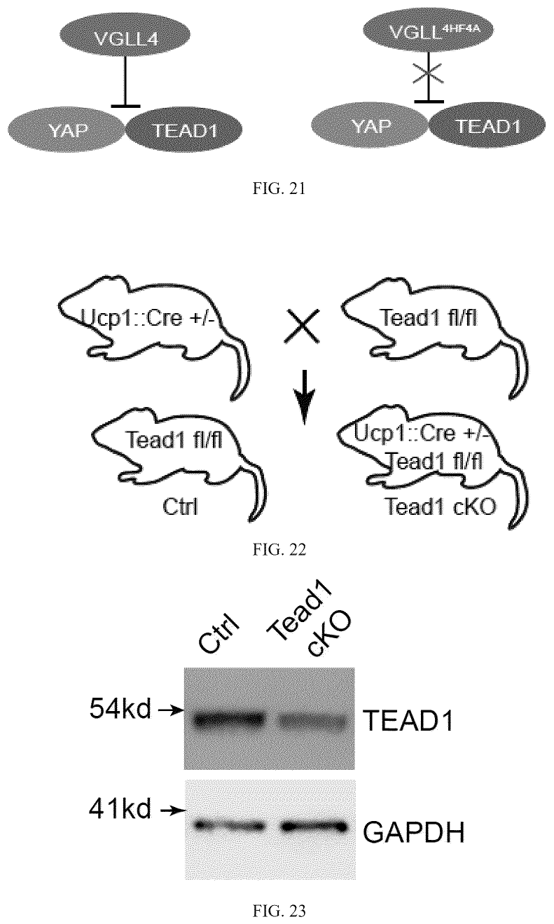

Each VGLL4 TDU domain has two essential animal acid residuals (HF) mediating VGLL4-TEAD interaction (SEQ ID NO: 41 and SEQ ID NO: 42, respectively). Replacing the HFs in the TDU domains with four alanine residues (FIG. 20) minimizes the interaction between VGLL4 and TEAD. Unlike VGLL$, VGLL-HF4A does not suppress a YAP-TEAD complex (FIG. 21). YAP/TAZ may promote BAT thermogenesis, raising the possibility of manipulating this pathway to reduce obesity.

### Example 3. TEAD1 Directly Regulates the Expression of Fgf21

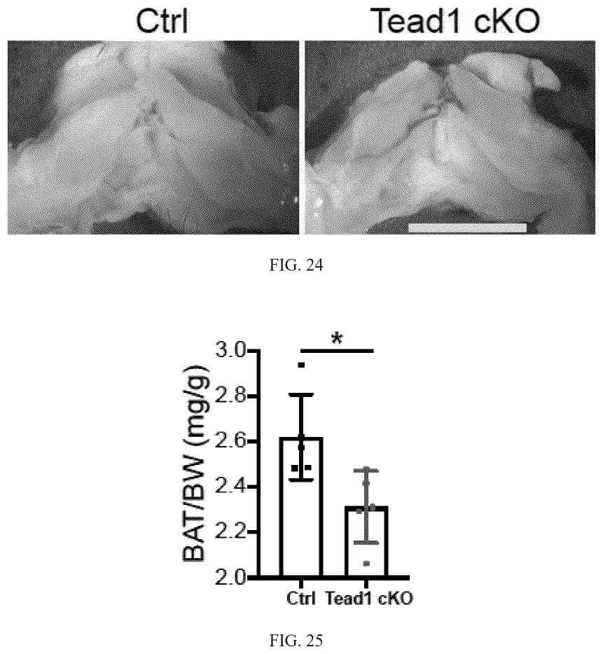

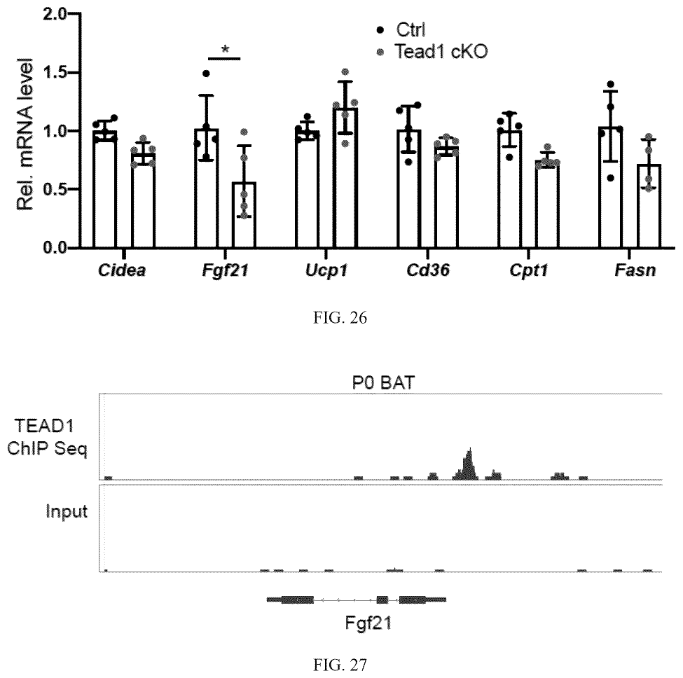

Double heterozygous YAP and TAZ knockout mice have previously been shown to have much smaller BAT than their littermate controls at four weeks after birth. In the Hippo-YAP pathway, YAP/TAZ interacts with TEAD proteins to regulate downstream targets expression. Thus, TEAD1 may regulated the postnatal growth of BAT. As disclosed herein, Ucp1::Cre transgenic mice were crossed with Tead1 flox allele to specifically delete Tead in the BAT (FIG. 22). TEAD1 depletion in BAT of Tead1 cKO mice was confirmed by western blot (FIG. 23). Compared with controls, the Tead1 cKO mice had smaller interscapular BAT deposits (FIGS. 24 and 25). Knocking out TEAD1 in the brown adipocytes significantly decreased the expression of Fgf21 in BAT (FIG. 26). Chromatin immunoprecipitation sequencing data demonstrated that TEAD1 directly binds to the promoter region of Fgf21 (FIG. 27). Fibroblast growth factor 21 (FGF21) is an important myokine that regulates glucose-lipid metabolism.

### Example 4. Activation of VGLL4 Reduces Adiposity

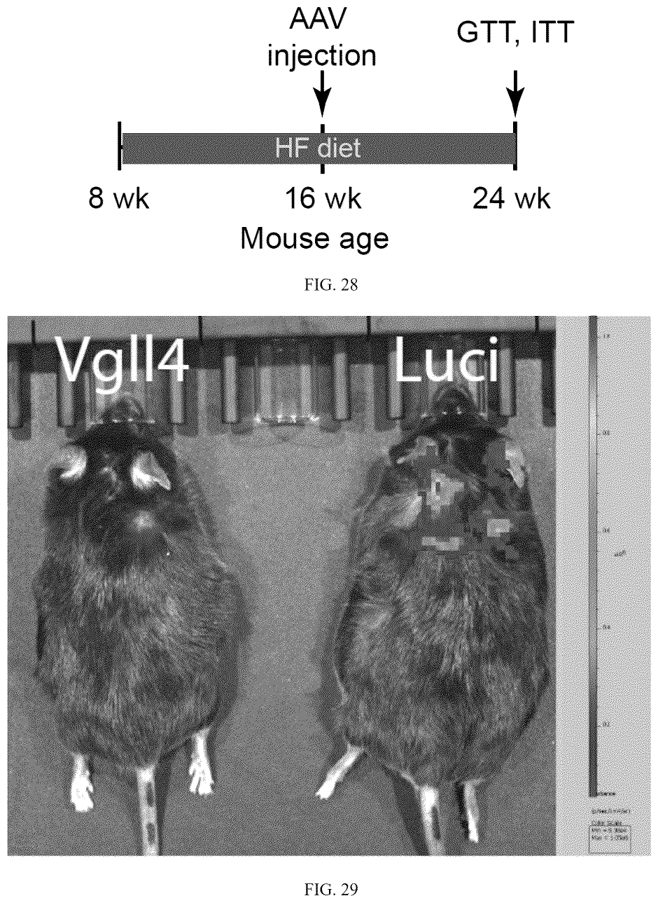

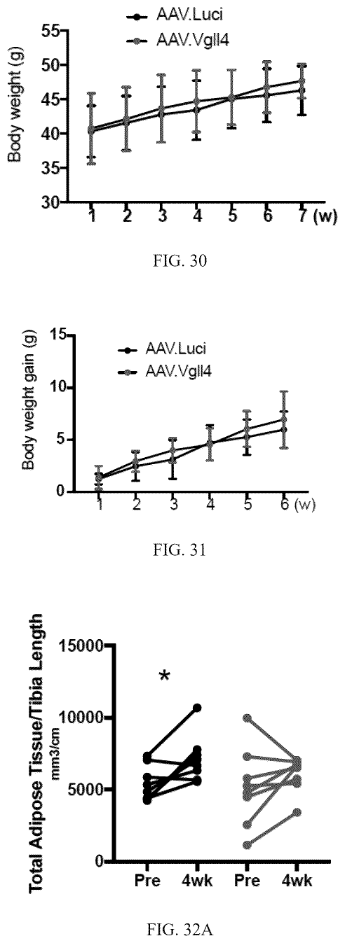

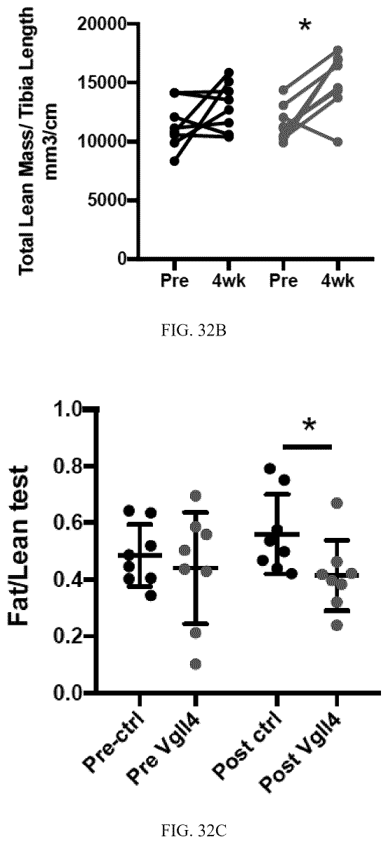

BAT plays important roles in non-shivering thermogenesis and energy homeostasis. As disclosed herein, AAV-mediated overexpression of VGLL4 increased BAT volume. To demonstrate whether activation of VGLL4 in an obesity model (mice fed on a high fat diet) would reduce body weight, AAV.BCE. VGLL4 (including SEQ ID NO: 39) into high fat diet induced obesity mice, and their body weight monitored for 7 weeks (FIG. 28). Controls received AAV with luciferase controlled by the cis regulatory element (SEQ ID NO: 3). 8 weeks after AAV delivery, luciferase signals were easily detected in the AAV.BCE.luci transduced mice (FIG. 29). Body weight and body weight gain values were not distinguishable between control and VGLL4 treated mice (FIGS. 30 and 31). However, using micro CT, the volume of adipose and non-adipose tissue (lean mass) were measured. 4 weeks after AAV transduction, although the body weight gain was similar between control and VGLL4 mice, the control but not the VGLL4 mice showed a significantly increase in adipose tissue mass. Meanwhile, VGLL4 but not the control mice had a significant increase of lean mass. Consequently, 4 weeks after AAV infusion, the VGLL4 mice had a lower fat/lean test ratio than the control mice (FIGS. 32A-C). Western blot showed that exogenous VGLL4 was expressed in BAT of AAV.BCE. VGLL4 transduced mice (not shown).

### Example 5. AAV.BCE. VGLL4 Mitigates Body Weight Gain

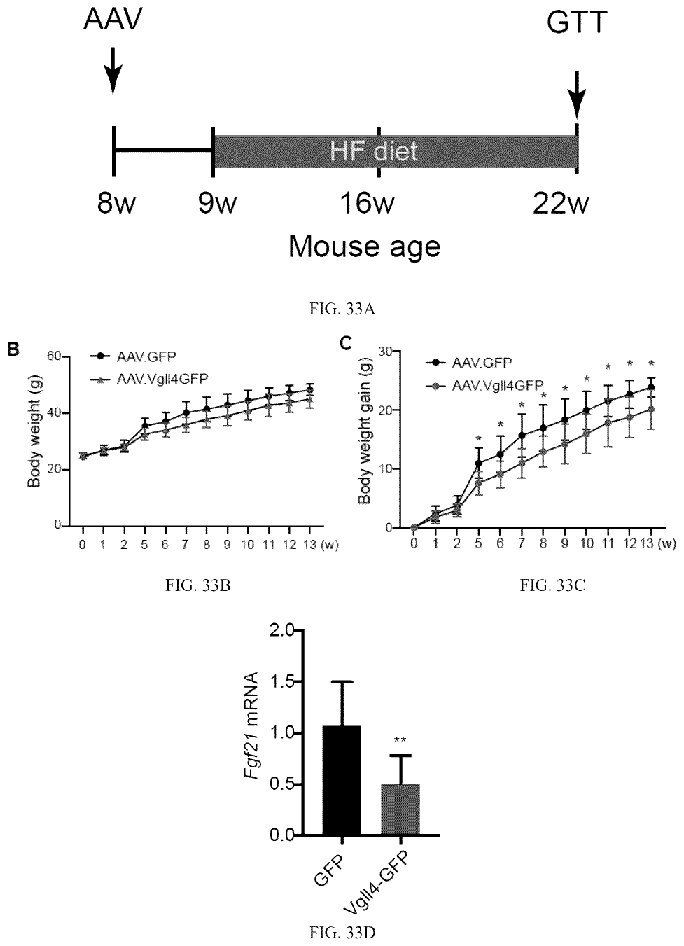

To demonstrate whether activation of VGLL4 in normal mice would prevent or mitigate the progression of obesity, AAV.BCE.VGLL4 (including SEQ ID NO: 39) was administered to 8-week-old mice. Beginning 1 week after injection, mice were fed a high-fat diet (FIG. 33A). At the end of 13 weeks high fat diet treatment, the body weight of VGLL4 mice was lower than the control mice, though the difference did not reach statistical significance (FIG. 33B). Starting from week 5, however, VGLL4 mice had a significantly lower accumulated body weight gain than controls (FIG. 33C). As disclosed herein (Example 2), AAV.BCE. VGLL4 targets both BAT and liver when administered later in development such as in adulthood. Fgf21 is a target of TEAD1 (FIGS. 26 and 27), and is mainly produced by liver and adipose tissue. Expression of VGLL4 and Fgf21 was therefore measure in liver. VGLL4 was overexpressed in liver, and that Fgf21 was significantly decreased in liver of VGLL4 mice (FIG. 33D).

### Example 6. AAV.BCE.VGLL4HF4A Increases BAT Mitochondrial Genes Expression

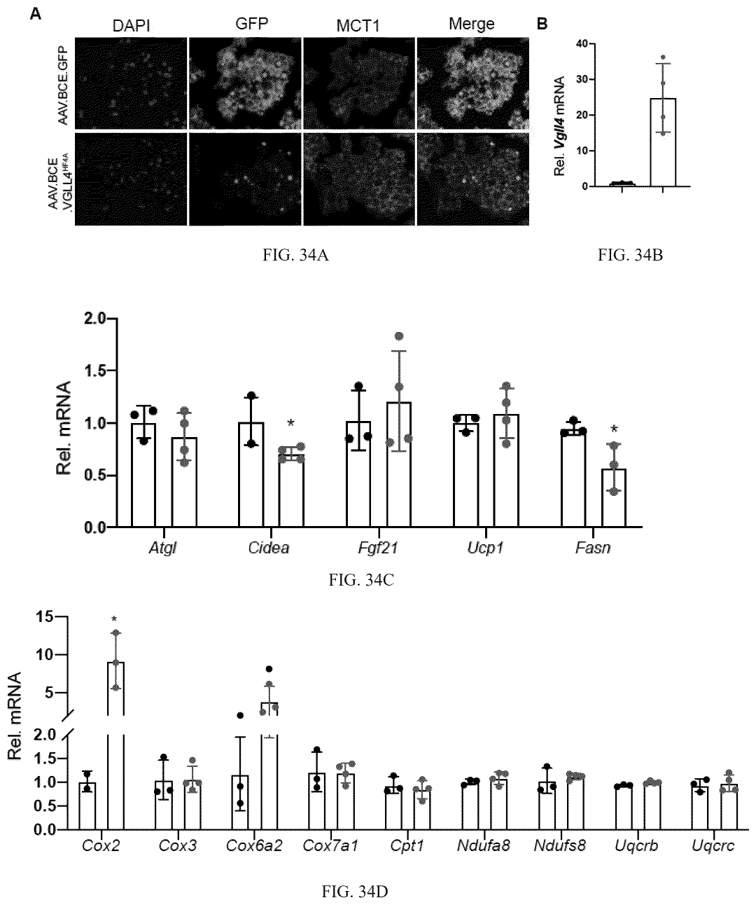

As disclosed herein, VGLL4 expression driven by a Ucp1 cis regulatory element mitigated body weight gain, it also suppressed the expression of Fgf21, which is important for glucose metabolism. VGLL4 may therefore have multiple roles, interacting with TEAD1 to decrease Fgf21 expression, while also interacting with other unknown factors to improve energy expenditure. Without being limited to any particular theory or mechanism of action, this possibility may indicate why VGLL4 may mitigate body weight gain without improving glucose metabolism. An AAV.BCE.VGLL4HF4A vector was created, which expresses a mutated VGLL4 that does not interact with TEAD (HF4A mutations), including SEQ ID NO: 36, and also including a GFP reporter protein (SEQ ID NO: 40). 8-week-old normal mice received subcutaneous injection of AAV.BCE. VGLL4HF4A, resulting in transfection of BAT (FIGS. 34A and B). qRT-PCR results showed that over-expression of VGLLAHF4A did not affect the expression of Fgf21 but reduced the expression of Cidea and Fasn (FIG. 34C), which are two genes involved in lipogenesis, in BAT. Additionally, VGLL4HF4A increased the expression of Cox2 (Cytochrome C Oxidase Subunit II, encoded by MT-CO2) and Cox6a2 (Cytochrome C Oxidase Subunit 6A2) in BAT (FIG. 34D). These data indicate that VGLLAHF4A may increase mitochondrial respiration activity without affecting Fgf21 expression, and also reduce lipogenesis.

### Example 7. AAV.BCE.VGLL4HF4A Mitigates Body Weight Gain and Reduces Serum Glucose Level

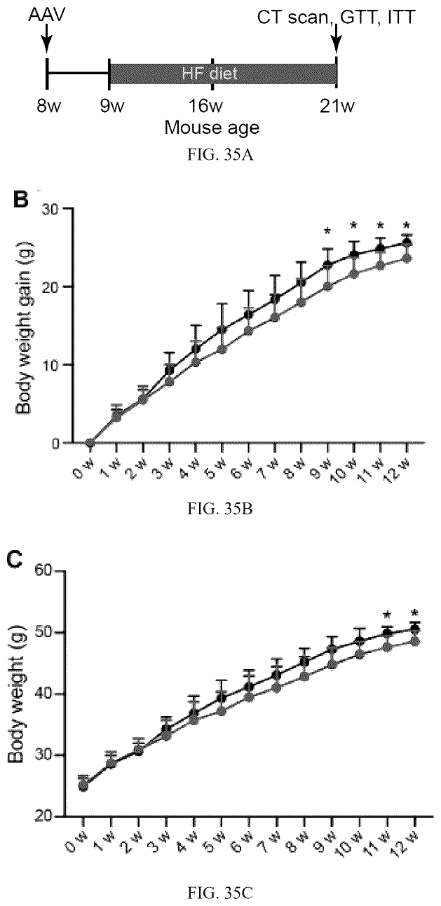

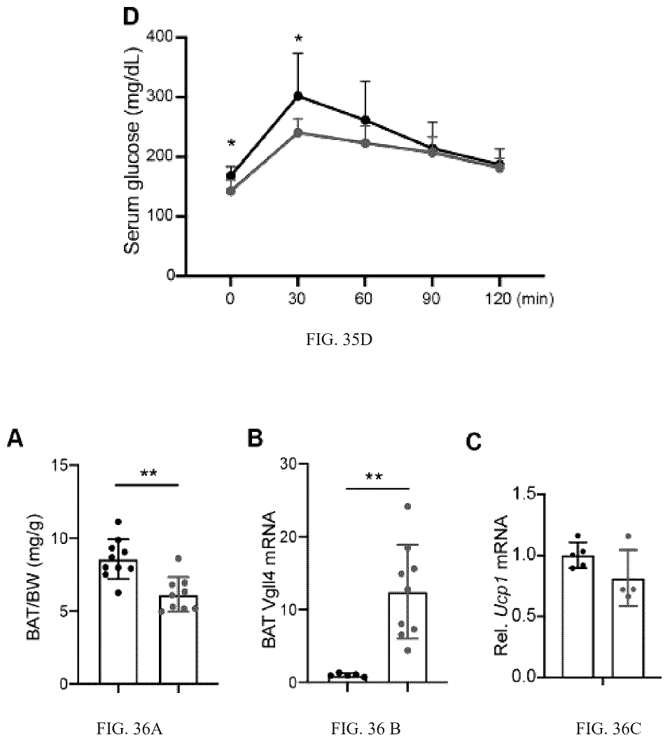

AAV.BCE.VGLLAHF4A (including SEQ ID NO: 36) was injected se to the inter-scapular region of 8 week old C57/BL6 mice, at a dosage of 2×10\acute{ }9 GC/gram body weight. AAV.BCE.GFP was used as control. One week after virus injection, 12 weeks of feeding with high fat diet (HFD) began (FIG. 35A). During HFD treatment, body weight gain rate of AAV.BCE.VGLL4HF4A mice (VGLL4HF4A) was slower than that of the AAV.BCE.GFP mice, and the difference reached to significance at 9 weeks after HFD treatment (FIG. 35B). 11 weeks after high fat diet treatment, the body weight of AAV.BCE.VGLLAHF4A mice started to become significantly lower than that of the AAV.BCE.GFP mice (FIG. 35C). A glucose tolerance test (GTT) showed that the starving serum glucose level and glucose peak level following glucose challenge was significantly lower in the AAV.BCE. VGLLAHF4A mice (FIG. 35D).

### Example 8. AAV.BCE.VGLL4HF4A Reduces BAT Weight

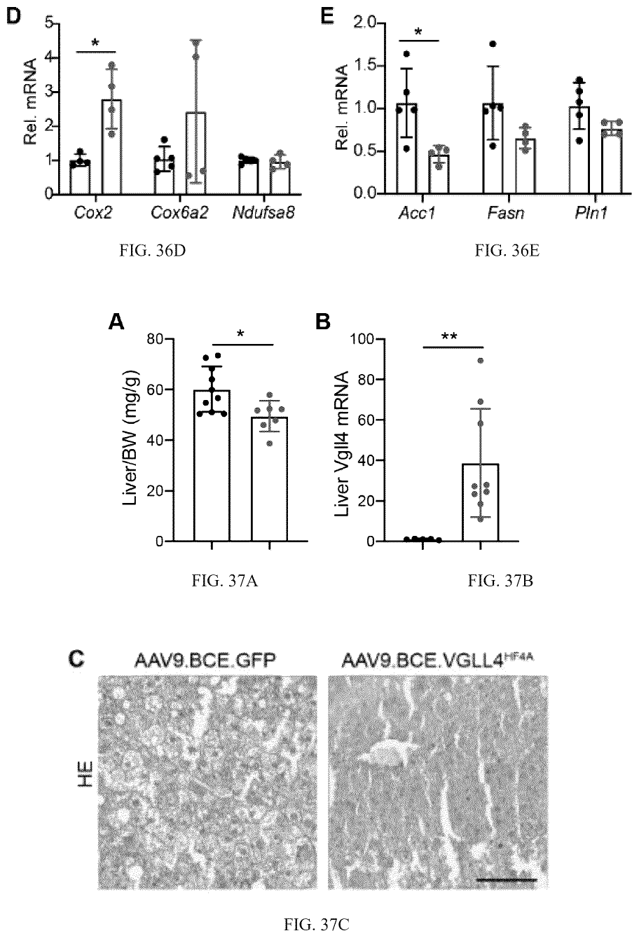

Compared to that of the GFP control mice, the mass of BAT was significantly lower in the VGLL4HF4A mice (FIG. 36A). qRT-PCR confirmed that VGLL4 was overexpressed in the BAT of VGLLAHF4A mice (FIG. 36B). The expression of Ucp1 was not affected by VGLL4HF4A (FIG. 36C). Expression levels of three more genes that regulate mitochondria respiration activity were also measured in BAT: Cox2, Cox6a, Ndufsa8. Cox2 was significantly upregulated in the VGLL4HF4A BAT (FIG. 36D). VGLL4HF4A also suppressed expression of Acc1, a gene involved in fatty acid synthesis (FIG. 36E). VGLLAHF4A may therefore preserve BAT function by both increasing the mitochondria respiration activity and attenuating fatty acid synthesis.

### Example 9. AAV.BCE.VGLL4HF4A Reduces Liver Weight and Fatty Acid Synthesis

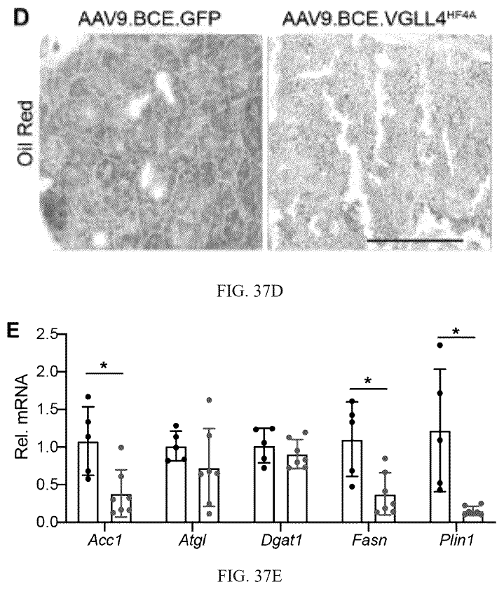

A BCE cis regulatory element including a Ucp1 enhancer and Ucp1 promoter as disclosed herein drives gene expression in liver when adult subjects are transfected, as disclosed herein. Interestingly, liver weight of VGLL4HF4A mice was significantly lower than that of the GFP control mice (FIG. 37A). qRT-PCR confirmed that VGLL4 was robustly overexpressed in liver of VGLL4HF4A mice (FIG. 37B). Histology determined by haematoxylin and eosin (H&E) staining showed moderate lipid droplets accumulation and microsteatosis (FIG. 37C). Oil red staining confirmed that VGLL4HF4A liver had much less lipid droplets accumulation than the GFP control liver (FIG. 37D). The expression of fatty acid synthesis genes Acc1 and Fasn in liver were significantly reduced by VGLL4HF4A (FIG. 37E). VGLL4HF4A may prevent HFD induced liver pathologies such as liver metabolic dysfunction, inflammation, or nonalcoholic fatty liver disease

It should be appreciated that all combinations of the foregoing concepts and additional concepts discussed in greater detail herein (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein and may be used to achieve the benefits and advantages described herein.

Although examples have been depicted and described in detail herein, it will be apparent to those skilled in the relevant art that various modifications, additions, substitutions, and the like can be made without departing from the spirit of the present disclosure and these are therefore considered to be within the scope of the present disclosure as defined in the claims that follow.

1-63. (canceled)

64. A polynucleotide, comprising
a nucleotide sequence encoding a vestigial like 4 protein, wherein the vestigial like 4 protein comprises a first TDU domain having a first amino acid sequence and a second TDU domain having a second amino acid sequence,
the first amino acid sequence comprises the amino acid sequence as set forth in SEQ ID NO: 41 with two amino acid substitutions, wherein the seventh amino acid of the amino acid sequence as set forth in SEQ ID NO: 41 is not H and the eighth amino acid of the amino acid sequence as set forth in SEQ ID NO: 41 is not F, and
the second amino acid sequence comprises the amino acid sequence as set forth in SEQ ID NO: 42, wherein the seventh amino acid of the amino acid sequence as set forth in SEQ ID NO: 42 is not H and the eighth amino acid of the amino acid sequence as set forth in SEQ ID NO: 42 is not F; and
a means for promoting expression in brown adipose tissue cells.

65. The polynucleotide of claim 64, wherein the vestigial like 4 protein has at least 90% homology with a sequence selected from SEQ ID NO: 21, SEQ ID NO: 23, SEQ ID NO: 25, SEQ ID NO: 27, SEQ ID NO: 29, SEQ ID NO: 31, and SEQ ID NO: 33.

66. The polynucleotide of claim 64, wherein the sequence encoding a vestigial like 4 protein has at least 90% identity with a sequence selected from SEQ ID NO: 22, SEQ ID NO: 24, SEQ ID NO: 26, SEQ ID NO: 28, SEQ ID NO: 30, and SEQ ID NO: 32.

67. The polynucleotide of claim 64, wherein the vestigial like 4 protein has from 0 to 3 substitutions to a sequence selected from SEQ ID NO: 23, SEQ ID NO: 25, SEQ ID NO: 27, SEQ ID NO: 29, SEQ ID NO: 31, and SEQ ID NO: 33, wherein the substitutions are not in the first TDU domain or the second TDU domain.

68. The polynucleotide of claim 64, wherein the polynucleotide further comprises an intron.

69. The polynucleotide of claim 68, wherein the intron has at least 90% homology with SEQ ID NO: 34.

70. A viral vector comprising the polynucleotide of claim 64.

71. The viral vector of claim 70, wherein the viral vector comprises an adenoviral associated vector.

72. The viral vector of claim 70, wherein the vestigial like 4 protein has at least 90% homology with a sequence selected from SEQ ID NO: 21, SEQ ID NO: 23, SEQ ID NO: 25, SEQ ID NO: 27, SEQ ID NO: 29, SEQ ID NO: 31, and SEQ ID NO: 33.

73. The viral vector of claim 70, wherein the sequence encoding a vestigial like 4 protein has at least 90% identity with a sequence selected from SEQ ID NO: 22, SEQ ID NO: 24, SEQ ID NO: 26, SEQ ID NO: 28, SEQ ID NO: 30, and SEQ ID NO: 32.

74. The viral vector of claim 70, wherein the vestigial like 4 protein has from 0 to 3 substitutions to a sequence selected from SEQ ID NO: 23, SEQ ID NO: 25, SEQ ID NO: 27, SEQ ID NO: 29, SEQ ID NO: 31, and SEQ ID NO: 33, wherein the substitutions are not in the first TDU domain or the second TDU domain.

75. The viral vector of claim 70, wherein the polynucleotide further comprises an intron.

76. The viral vector of claim 70, wherein the intron has at least 90% homology with SEQ ID NO: 34.

77. The viral vector of claim 71, wherein the vestigial like 4 protein has at least 90% homology with a sequence selected from SEQ ID NO: 21, SEQ ID NO: 23, SEQ ID NO: 25, SEQ ID NO: 27, SEQ ID NO: 29, SEQ ID NO: 31, and SEQ ID NO: 33.

78. The viral vector of claim 71, wherein the sequence encoding a vestigial like 4 protein has at least 90% identity with a sequence selected from SEQ ID NO: 22, SEQ ID NO: 24, SEQ ID NO: 26, SEQ ID NO: 28, SEQ ID NO: 30, and SEQ ID NO: 32.

79. The viral vector of claim 71, wherein the vestigial like 4 protein has from 0 to 3 substitutions to a sequence selected from SEQ ID NO: 23, SEQ ID NO: 25, SEQ ID NO: 27, SEQ ID NO: 29, SEQ ID NO: 31, and SEQ ID NO: 33, wherein the substitutions are not in the first TDU domain or the second TDU domain.

80. The viral vector of claim 71, wherein the polynucleotide further comprises an intron.

81. The viral vector of claim 71, wherein the intron has at least 90% homology with SEQ ID NO: 34.

82. The polynucleotide of claim 64, wherein the seventh amino acid of the amino acid sequence as set forth in SEQ ID NO: 41 is A, the eighth amino acid of the amino acid sequence as set forth in SEQ ID NO: 41 is A, the seventh amino acid of the amino acid sequence as set forth in SEQ ID NO: 42 is A, and the eighth amino acid of the amino acid sequence as set forth in SEQ ID NO: 42 is A.

83. A viral vector, comprising the polynucleotide of claim 82.
